SEQUENCE LISTING (4 sequences)

SEQ ID NO: 1 - DNA, 1967 bp, synthetic construct
  source 1..1967: mol_type = other DNA
tcctggatgggaccacgctagagctcctaaataaaagaccagagatgagctgaccctgatccatttccaagagcctaaggtaggagaaatgtctgttgtgctctgatgctttccatggatgccatcatgatctgcatcccgaagggagccaagaaagatggtacccacacagctttcctcttgatgagtcatacagtctgaaagccttactctgctttatccacctggcagagatctattgagaatgtgtgttgtgtaggctcaagtacaagaaagcaggaacaaagagtttccagtgttggactttacttctatgcacagtgttgaaaacccatgtatgcaggaagcaaagaaaggtgtgagggtggtagacgacctgcttggggctcccgtgcctaaaaaaaaaaaaatgttgtcaaaattagccagccccttccacccaaaatttccatcaagatgggcacaaggaccctcatctcttgttcaccgagtagccattggttgctcacagccacttatgaccttcttacctctttctgatcttcctcattttctgccttattggtgctggccacataacaaatgctgtggtcttatacatggtgctctcaaccccagtgaccagtggttgataaggagtttctattttgagcacgttggttgtttctgacctggcctataggcatggctgctggggtcaacatctgaagggtgttccctaccatatgatgactaccacaagtggcatatgttctttatttttgagacagggtcttgctctgtagcccaggctggagtgcagtggtgcgatcatagctcgctgtagccttgaactcttgggctcaagcgatcctcccacctcagccttccaagtagctgggactacaagcacaagccactgtacctgactaatttttattttattttatttttgtagagacagagtctcactgtgttgcccaagctgatctcaaattcctggcctcaagcagtcctcccgcctcagcctcccaaagtgctgggattacagacatgagccaccacccaacctcatgttctaactgataaaacttacaagtttataaaacttataaaattctcagaaagtgaaaactgatttgagaaatgacctggatatgaaaaactaagtacagatcctttagcgacttacaagttggttgactttggtgacatcactttgtctctcttgaccactataaaagaagggtttggagagattcccagctatgggccaaggactagttacatcataatcacctggggccattgttaaaaagtcacattcccacaccttttcacagttctgaatcaatctcctgaatgggaatgttttcctagcaaacaatccaggtgattctcatgggcatccgggcttaggaaccactgtggcagatgatctcttctgatgcgaagaggtcactgagcttggctgtgttagccaggtttaggtggggctggaccgggtagggataggggactgtctgacctctcccagggactgtgttcagggaacccaagggaatctgagaaggaagagaaagttaccacctttgacacatgtggcagagttctctggaacctctcctctgttcctaccacaggaaggccacaggcacgcaccagggctgtactggcctaaaccatagttctgtgctgactgagaggttctgaaattagaaaagaggtctaaatcctgcttccaggtttttgtgaggtggagaaaagcagagtgacctgggagaagtggttatcaacaacacagctggtgacttcttccctggggtgaggaaggttttccctttccctgccccgagctgcagtggtgcagtctgttaccttggagaccagctaacacattcccgcagggcaagcttcgctgctgagttgcttggaggagcttggagaaaccagaagtgagatccaggagaagtaaggccctggagtgc

SEQ ID NO: 2 - DNA, 2118 bp, synthetic construct
  source 1..2118: mol_type = other DNA
tcctggatgggaccacgctagagctcctaaataaaagaccagagatgagctgaccctgatccatttccaagagcctaaggtaggagaaatgtctgttgtgctctgatgctttccatggatgccatcatgatctgcatcccgaagggagccaagaaagatggtacccacacagctttcctcttgatgagtcatacagtctgaaagccttactctgctttatccacctggcagagatctattgagaatgtgtgttgtgtaggctcaagtacaagaaagcaggaacaaagagtttccagtgttggactttacttctatgcacagtgttgaaaacccatgtatgcaggaagcaaagaaaggtgtgagggtggtagacgacctgcttggggctcccgtgcctaaaaaaaaaaaaatgttgtcaaaattagccagccccttccacccaaaatttccatcaagatgggcacaaggaccctcatctcttgttcaccgagtagccattggttgctcacagccacttatgaccttcttacctctttctgatcttcctcattttctgccttattggtgctggccacataacaaatgctgtggtcttatacatggtgctctcaaccccagtgaccagtggttgataaggagtttctattttgagcacgttggttgtttctgacctggcctataggcatggctgctggggtcaacatctgaagggtgttccctaccatatgatgactaccacaagtggcatatgttctttatttttgagacagggtcttgctctgtagcccaggctggagtgcagtggtgcgatcatagctcgctgtagccttgaactcttgggctcaagcgatcctcccacctcagccttccaagtagctgggactacaagcacaagccactgtacctgactaatttttattttattttatttttgtagagacagagtctcactgtgttgcccaagctgatctcaaattcctggcctcaagcagtcctcccgcctcagcctcccaaagtgctgggattacagacatgagccaccacccaacctcatgttctaactgataaaacttacaagtttataaaacttataaaattctcagaaagtgaaaactgatttgagaaatgacctggatatgaaaaactaagtacagatcctttagcgacttacaagttggttgactttggtgacatcactttgtctctcttgaccactataaaagaagggtttggagagattcccagctatgggccaaggactagttacatcataatcacctggggccattgttaaaaagtcacattcccacaccttttcacagttctgaatcaatctcctgaatgggaatgttttcctagcaaacaatccaggtgattctcatgggcatccgggcttaggaaccactgtggcagatgatctcttctgatgcgaagaggtcactgagcttggctgtgttagccaggtttaggtggggctggaccgggtagggataggggactgtctgacctctcccagggactgtgttcagggaacccaagggaatctgagaaggaagagaaagttaccacctttgacacatgtggcagagttctctggaacctctcctctgttcctaccacaggaaggccacaggcacgcaccagggctgtactggcctaaaccatagttctgtgctgactgagaggttctgaaattagaaaagaggtctaaatcctgcttccaggtttttgtgaggtggagaaaagcagagtgacctgggagaagtggttatcaacaacacagctggtgacttcttccctggggtgaggaaggttttccctttccctgccccgagctgcagtggtgcagtctgttaccttggagaccagctaacacattcccgcagggcaagcttcgctgctgagttgcttggaggagcttggagaaaccagaagtgagatccaggagaagtaaggccctggagtgccttgaggcaggtaagtatcaaggttacaagacaggtttaaggagaccaatagaaactgggcttgtcgagacagagaagactcttgcgtttctgataggcacctattggtcttactgacatccactttgcctttctctccacaggtgtccac

SEQ ID NO: 3 - DNA, 2932 bp, synthetic construct
  source 1..2932: mol_type = other DNA
tcctggatgggaccacgctagagctcctaaataaaagaccagagatgagctgaccctgatccatttccaagagcctaaggtaggagaaatgtctgttgtgctctgatgctttccatggatgccatcatgatctgcatcccgaagggagccaagaaagatggtacccacacagctttcctcttgatgagtcatacagtctgaaagccttactctgctttatccacctggcagagatctattgagaatgtgtgttgtgtaggctcaagtacaagaaagcaggaacaaagagtttccagtgttggactttacttctatgcacagtgttgaaaacccatgtatgcaggaagcaaagaaaggtgtgagggtggtagacgacctgcttggggctcccgtgcctaaaaaaaaaaaaatgttgtcaaaattagccagccccttccacccaaaatttccatcaagatgggcacaaggaccctcatctcttgttcaccgagtagccattggttgctcacagccacttatgaccttcttacctctttctgatcttcctcattttctgccttattggtgctggccacataacaaatgctgtggtcttatacatggtgctctcaaccccagtgaccagtggttgataaggagtttctattttgagcacgttggttgtttctgacctggcctataggcatggctgctggggtcaacatctgaagggtgttccctaccatatgatgactaccacaagtggcatatgttctttatttttgagacagggtcttgctctgtagcccaggctggagtgcagtggtgcgatcatagctcgctgtagccttgaactcttgggctcaagcgatcctcccacctcagccttccaagtagctgggactacaagcacaagccactgtacctgactaatttttattttattttatttttgtagagacagagtctcactgtgttgcccaagctgatctcaaattcctggcctcaagcagtcctcccgcctcagcctcccaaagtgctgggattacagacatgagccaccacccaacctcatgttctaactgataaaacttacaagtttataaaacttataaaattctcagaaagtgaaaactgatttgagaaatgacctggatatgaaaaactaagtacagatcctttagcgacttacaagttggttgactttggtgacatcactttgtctctcttgaccactataaaagaagggtttggagagattcccagctatgggccaaggactagttacatcataatcacctggggccattgttaaaaagtcacattcccacaccttttcacagttctgaatcaatctcctgaatgggaatgttttcctagcaaacaatccaggtgattctcatgggcatccgggcttaggaaccactgtggcagatgatctcttctgatgcgaagaggtcactgagcttggctgtgttagccaggtttaggtggggctggaccgggtagggataggggactgtctgacctctcccagggactgtgttcagggaacccaagggaatctgagaaggaagagaaagttaccacctttgacacatgtggcagagttctctggaacctctcctctgttcctaccacaggaaggccacaggcacgcaccagggctgtactggcctaaaccatagttctgtgctgactgagaggttctgaaattagaaaagaggtctaaatcctgcttccaggtttttgtgaggtggagaaaagcagagtgacctgggagaagtggttatcaacaacacagctggtgacttcttccctggggtgaggaaggttttccctttccctgccccgagctgcagtggtgcagtctgttaccttggagaccagctaacacattcccgcagggcaagcttcgctgctgagttgcttggaggagcttggagaaaccagaagtgagatccaggagaagtaaggccctggagtgcctcgagctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctggg

SEQ ID NO: 4 - DNA, 3742 bp, synthetic construct
  source 1..3742: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctacgcgtgtctgtctgcacatttcgtagagcgagtgttccgatactctaatctccctaggactagttcctggatgggaccacgctagagctcctaaataaaagaccagagatgagctgaccctgatccatttccaagagcctaaggtaggagaaatgtctgttgtgctctgatgctttccatggatgccatcatgatctgcatcccgaagggagccaagaaagatggtacccacacagctttcctcttgatgagtcatacagtctgaaagccttactctgctttatccacctggcagagatctattgagaatgtgtgttgtgtaggctcaagtacaagaaagcaggaacaaagagtttccagtgttggactttacttctatgcacagtgttgaaaacccatgtatgcaggaagcaaagaaaggtgtgagggtggtagacgacctgcttggggctcccgtgcctaaaaaaaaaaaaatgttgtcaaaattagccagccccttccacccaaaatttccatcaagatgggcacaaggaccctcatctcttgttcaccgagtagccattggttgctcacagccacttatgaccttcttacctctttctgatcttcctcattttctgccttattggtgctggccacataacaaatgctgtggtcttatacatggtgctctcaaccccagtgaccagtggttgataaggagtttctattttgagcacgttggttgtttctgacctggcctataggcatggctgctggggtcaacatctgaagggtgttccctaccatatgatgactaccacaagtggcatatgttctttatttttgagacagggtcttgctctgtagcccaggctggagtgcagtggtgcgatcatagctcgctgtagccttgaactcttgggctcaagcgatcctcccacctcagccttccaagtagctgggactacaagcacaagccactgtacctgactaatttttattttattttatttttgtagagacagagtctcactgtgttgcccaagctgatctcaaattcctggcctcaagcagtcctcccgcctcagcctcccaaagtgctgggattacagacatgagccaccacccaacctcatgttctaactgataaaacttacaagtttataaaacttataaaattctcagaaagtgaaaactgatttgagaaatgacctggatatgaaaaactaagtacagatcctttagcgacttacaagttggttgactttggtgacatcactttgtctctcttgaccactataaaagaagggtttggagagattcccagctatgggccaaggactagttacatcataatcacctggggccattgttaaaaagtcacattcccacaccttttcacagttctgaatcaatctcctgaatgggaatgttttcctagcaaacaatccaggtgattctcatgggcatccgggcttaggaaccactgtggcagatgatctcttctgatgcgaagaggtcactgagcttggctgtgttagccaggtttaggtggggctggaccgggtagggataggggactgtctgacctctcccagggactgtgttcagggaacccaagggaatctgagaaggaagagaaagttaccacctttgacacatgtggcagagttctctggaacctctcctctgttcctaccacaggaaggccacaggcacgcaccagggctgtactggcctaaaccatagttctgtgctgactgagaggttctgaaattagaaaagaggtctaaatcctgcttccaggtttttgtgaggtggagaaaagcagagtgacctgggagaagtggttatcaacaacacagctggtgacttcttccctggggtgaggaaggttttccctttccctgccccgagctgcagtggtgcagtctgttaccttggagaccagctaacacattcccgcagggcaagcttcgctgctgagttgcttggaggagcttggagaaaccagaagtgagatccaggagaagtaaggccctggagtgcctcgaggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcgaggccaccatgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagtgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactgaggtacccaattcgccctatagtgagtcgtgctagctgcaggatctcgatatcagcggccgcactagacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaatctagagatctgtgtgttggttttttgtgtaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

(19) United States
(12) Patent Application Publication
(10) Pub. No.: US 2025/0269064 A1
(43) Pub. Date: Aug. 28, 2025

(54) HUMAN EPENDYMA-SPECIFIC PROMOTER AND USES THEREOF

(71) Applicants: THE CHILDREN'S HOSPITAL OF PHILADELPHIA, Philadelphia, PA (US); THE GENERAL HOSPITAL CORPORATION, Boston, MA (US)

(72) Inventors: Beverly DAVISON, Philadelphia, PA (US); Megan KEISER, Philadelphia, PA (US); Yonghong CHEN, Philadelphia, PA (US); Ellie CARRELL, Philadelphia, PA (US); Bradley HYMAN, Boston, MA (US); Rosemary Joan JACKSON, Boston, MA (US)

(73) Assignees: THE CHILDREN'S HOSPITAL OF PHILADELPHIA, Philadelphia, PA (US); THE GENERAL HOSPITAL CORPORATION, Boston, MA (US)

(21) Appl. No.: 18/858,251

(22) PCT Filed: Apr. 21, 2023
(86) PCT No.: PCT/US2023/019371

§ 371 Date: Oct. 18, 2024

Related U.S. Application Data
(60) Provisional application No. 63/381,689, filed on Oct. 31, 2022.
(60) Provisional application No. 63/482,155, filed on Jan. 30, 2023.
(60) Provisional application No. 63/333,979, filed on Apr. 22, 2022.

(51) Int. Cl. A61K 48/00; A61K 38/17; A61P 25/28

(52) U.S. Cl. CPC A61K 48/0058; A61K 38/1709; A61K 48/0066; A61K 48/0075; A61P 25/28

(57) ABSTRACT

Provided herein are compositions and methods for delivering a molecular therapeutic to the ependyma of a subject. The methods comprise administering an adeno-associated virus (AAV) to the subject. The AAVs encode a therapeutic transgene under the control of an ependyma-specific promoter.

### PRIORITY CLAIM

This application claims benefit of priority to U.S. Provisional Application Ser. Nos. 63/482,155, 63/381,689 and 63/333,979 filed Jan. 30, 2023, Oct. 31, 2022, Apr. 22, 2022, and Jan. 30, 2023, respectively, the entire contents of each application being hereby incorporated by reference.

### BACKGROUND

### 1. Field

The present disclosure relates generally to the fields of medicine and virology. More particularly, it concerns compositions and methods for delivery of molecular therapeutics to patients, particularly to the brain or central nervous system.

### 2. Description of Related Art

Adeno-associated viruses (AAVs) represent strong therapeutic candidates for the treatment of neurological disease. AAVs are non-enveloped, single-stranded DNA viruses that can infect both dividing and non-dividing cells. Following infection, the virus does not exhibit robust integration within the host genome but persists as an episome in the cell nucleus. Expression of AAV cargoes is controlled spatially at the level of the packaging capsid and by the transgene promoter. And because use of AAV for the treatment of disease may necessitate intervention in diseased tissue, a problem can arise in that target tissue that contains a different gene expression profile than its healthy counterpart. Finding the correct promoter sequence to drive therapeutic transgene expression is an important goal.

### SUMMARY

Provided herein are methods of expressing a therapeutic transgene in ependymal tissue of a subject, comprising administering to the subject a modified adeno-associated virus (AAV) encoding a therapeutic transgene under the control of a promoter selected from the group consisting of SEQ ID NO: 1, SEQ ID NO: 2, SEQ ID NO: 3, or a promoter having at least about 80% sequence identity therewith. The promoter may comprise or consist of SEQ ID NO: 1 or a promoter having at least about 85%, 90%, 95%, 96%, 97%, 98% or 99% sequence identity therewith. The promoter may comprise or consist of SEQ ID NO: 2 or a promoter having at least about 85%, 90%, 95%, 96%, 97%, 98% or 99% sequence identity therewith. The promoter may comprise or consist of SEQ ID NO: 3 or a promoter having at least about 85% t, 90%, 95%, 96%, 97%, 98% or 99% sequence identity therewith.

The modified AAV may comprise a modified capsid protein, such as where the modified capsid protein comprises a targeting peptide, wherein the targeting peptide is three to ten amino acids in length, such as seven amino acids in length. The modified AAV capsid protein may be a modified AAV1 capsid protein, a modified AAV2 capsid protein, or a modified AAV9 capsid protein. The modified AAV capsid protein may be derived from an AAV1 capsid protein, wherein the targeting peptide is inserted after residue 590 of the AAV1 capsid protein.

The targeting peptide may be flanked by linker sequences, wherein the linker sequences on each side of the targeting peptides are two or three amino acids long, such as where the sequences are SSA on the N-terminal side of the targeting peptide and AS on the C-terminal side of the targeting peptide.

The modified AAV capsid protein may be derived from an AAV2 capsid protein, wherein the targeting peptide is inserted after residue 587 of the AAV2 capsid protein. The targeting peptide may be flanked by linker sequences, wherein the linker sequences on each side of the targeting peptides are two or three amino acids long, such as where the linker sequences are AAA on the N-terminal side of the targeting peptide and AA on the C-terminal side of the targeting peptide.

The modified AAV capsid protein may be derived from an AAV9 capsid protein, wherein the targeting peptide is inserted after residue 588 of the AAV9 capsid protein. The targeting peptide is flanked by linker sequences, wherein the linker sequences on each side of the targeting peptides are two or three amino acids long, such as where the linker sequences are AAA on the N-terminal side of the targeting peptide and AS on the C-terminal side of the targeting peptide.

The therapeutic transgene may be an siRNA, shRNA, miRNA, non-coding RNA, lncRNA, therapeutic protein, or CRISPR system. The therapeutic transgene may be ApoE2 and the subject suffers from or is at an increased risk of developing Alzheimer's Disease as compared to the populational average. Administration may be direct intracerebroventricular or intraparenchymal injection. The modified AAV may be administered more than once, such as 2, 3, 4, 5, 6, 7, 8, 9, 10 or more times. The modified AAV may be administered monthly, every other month, every three months, every four months, every six months or annually. The method may further comprise providing a non-AAV therapy to said subject.

The method may comprise administration of a plurality of viral particles, such as wherein the virus is administered at a dose of about 1×10⁶ to about 1×10¹⁸ vector genomes per kilogram (vg/kg), or wherein the virus is administered at a dose from about 1×10⁷-1×10¹⁷, about 1×10⁸-1×10¹⁶, about 1×10⁹-1×10¹⁵, about 1×10¹⁰-1×10¹⁴, about 1×10¹⁰-1×10¹³, about 1×10¹⁰-1×10¹³, about 1×10¹⁰-1×10¹¹, about 1×10¹¹-1×10¹², about 1×10¹²-1×10¹³, or about 1×10¹³-1×10¹⁴ vg/kg of the subject. The subject may be human or a non-human mammal. The human subject may be 50 or more years old. The therapeutic transgene may be linked to a poly-adenylation signal.

Also provided is a modified adeno-associated virus (AAV) encoding a therapeutic transgene operably linked to a promoter selected from the group consisting of SEQ ID NO: 1, SEQ ID NO: 2, SEQ ID NO: 3, or a promoter having at least about 80% sequence identity therewith. The promoter may comprise or consist of SEQ ID NO: 1 or a promoter having at least about 85%, 90%, 95%, 96%, 97%, 98% or 99% sequence identity therewith. The promoter may comprise or consist of SEQ ID NO: 2 or a promoter having at least about 85%, 90%, 95%, 96%, 97%, 98% or 99% sequence identity therewith. The promoter may comprise of consist of SEQ ID NO: 3 or a promoter having at least about 85%, 90%, 95%, 96%, 97%, 98% or 99% sequence identity therewith.

The modified AAV may comprise a modified capsid protein, such as wherein the modified capsid protein comprises a targeting peptide, wherein the targeting peptide is three to ten amino acids in length, such as seven amino acids in length. The modified AAV capsid protein may be a modified AAV1 capsid protein, a modified AAV2 capsid protein, or a modified AAV9 capsid protein.

The modified AAV capsid protein may be derived from an AAV1 capsid protein, wherein the targeting peptide is inserted after residue 590 of the AAV1 capsid protein, such as wherein the targeting peptide is flanked by linker sequences, wherein the linker sequences on each side of the targeting peptides are two or three amino acids long. The linker sequences may be SSA on the N-terminal side of the targeting peptide and AS on the C-terminal side of the targeting peptide.

The modified AAV capsid protein may be derived from an AAV2 capsid protein, wherein the targeting peptide is inserted after residue 587 of the AAV2 capsid protein, such as wherein the targeting peptide is flanked by linker sequences, wherein the linker sequences on each side of the targeting peptides are two or three amino acids long. The linker sequences may be AAA on the N-terminal side of the targeting peptide and AA on the C-terminal side of the targeting peptide.

The modified AAV capsid protein may be derived from an AAV9 capsid protein, wherein the targeting peptide is inserted after residue 588 of the AAV9 capsid protein, such as wherein the targeting peptide is flanked by linker sequences, wherein the linker sequences on each side of the targeting peptides are two or three amino acids long. The linker sequences may be AAA on the N-terminal side of the targeting peptide and AS on the C-terminal side of the targeting peptide.

The therapeutic transgene may be an siRNA, shRNA, miRNA, non-coding RNA, lncRNA, therapeutic protein, or CRISPR system. The therapeutic transgene may be linked to a poly-adenylation signal. The therapeutic transgene may be transcriptionally linked to a detectable reporter, e.g., sequence encoding a fluorescent protein, a peptide tag, or a luciferase.

In another embodiment, there is provided a pharmaceutical composition comprising the modified AAV as described herein and a pharmaceutically acceptable carrier.

In yet another embodiment, there is provided an isolated and purified nucleic acid comprising a sequence selected from the group consisting of SEQ ID NO: 1, SEQ ID NO: 2, SEQ ID NO: 3, or a sequence having at least about 80% sequence identity therewith. The sequence may comprise or consist of SEQ ID NO: 1 or a sequence having at least about 85%, 90%, 95%, 96%, 97%, 98% or 99% sequence identity therewith. The sequence may comprise or consist of SEQ ID NO: 2 or a sequence having at least about 85%, 90%, 95%, 96%, 97%, 98% or 99% sequence identity therewith. The sequence may comprise or consist of SEQ ID NO: 3 or a sequence having at least about 85%, 90%, 95%, 96%, 97%, 98% or 99% sequence identity therewith.

The sequence may be operably connected to a heterologous coding region. The nucleic acid may further comprise one of more of (a) a multipurpose cloning site, (b) a transcription termination signal, (c) a poly-adenylation sequence, and/or (d) an origin of replication. The nucleic acid may further comprise one of more of (a) a sequence encoding a detectable marker, (b) a sequence encoding an affinity tag, and/or (c) one or two adeno-associated virus inverted terminal repeats. The nucleic acid may be contained in a replicable vector. The therapeutic transgene may be transcriptionally linked to a reporter by a sequence encoding a 2A “self-cleaving” peptide.

In still another embodiment, there is provided a method of reducing or impairing microglial inflammation comprise delivering ApoE2 to microglia in a subject in need thereof. The delivering of ApoE2 to the microglial comprises administering to said subject a modified AAV as defined herein or a pharmaceutical formulation comprising the same, wherein the therapeutic transgene is ApoE2. The microglial inflammation may be caused by or associated with a neurodegenerative disease, such as Huntington's disease, Parkinson's disease, motor neuron disease, spinocerebellar ataxia, spinal muscular atrophy, progressive supranuclear palsy, amyotrophic lateral sclerosis, multiple sclerosis, Batten disease, and Creutzfeldt-Jakob disease. Microglial inflammation may be caused by or associated with Alzheimer's disease.

The administration may be by direct intracerebroventricular or intraparenchymal injection of ApoE2 or a modified AAV, and may involve more than one administration, such as 2, 3, 4, 5, 6, 7, 8, 9, 10 or more times, and/or monthly, every other month, every three months, every four months, every six months or annually. The method may further comprise providing a non-AAV ApoE2 therapy to said subject.

For AAV therapy, a plurality of viral particles may be administered, such as at a dose of about 1×10⁶ to about 1×10¹⁸ vector genomes per kilogram (vg/kg), including at a dose from about 1×10⁷-1×10¹⁷, about 1×10⁸-1×10¹⁶, about 1×10⁹-1×10¹⁵, about 1×10¹⁰-1×10¹⁴, about 1×10¹⁰-1×10¹³, about 1×10¹⁰-1×10¹³, about 1×10¹⁰-1×10¹, about 1×10¹¹-1×10¹², about 1×10¹²-1×10¹³, or about 1×10¹³-1×10¹⁴ vg/kg of the patient. The subject may be human, a non-human mammal, or a human subject 50 or more years old. The therapeutic transgene may be linked to a poly-adenylation signal.

Other objects, features and advantages of the present disclosure will become apparent from the following detailed description. It should be understood, however, that the detailed description and the specific examples, while indicating preferred embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present disclosure. The disclosure may be better understood by reference to one or more of these drawings in combination with the detailed description of specific embodiments presented herein.

FIG. 1. Identify ependyma-specific promoters. Due to the thin nature of the ependymal lining of the ventricles, the inventors employed a subtractive approach to identify tissue-specific genes. Samples were obtained from ventricle-adjacent white matter and grey matter, as well as regions at the ventricle margin that included white and grey matter along with ependyma. Genes unique to the white matter+ependyma and grey matter+ependyma samples were categorized as enriched. To ensure active promoter use in multiple relevant disease states, samples were sourced from healthy controls as well as from Alzheimer's disease (AD), Huntington's Disease (HD), Frontotemporal Dementia, Lewy body dementia, sematic dementia, and dementia patients.

FIG. 2. Validate expression in ependyma. Top gene hits were validated against published in situ hybridization data from the Allen Brain Institute. − is not ependyma-specific/enriched; + od ependyma specific/enriched; NA no data available.

FIG. 3. Validate expression in ependyma. Cartoon diagram of ependyma-specific promoter transgenes. Approximately 1100-2500 bp of potential promoter sequence from ependyma-enriched genes was cloned upstream an eGFP reporter. Individual transgenes were identified using a 3-bp barcode in the 3′UTR of the RNA transcript.

FIG. 4. hEpendyma promoter library use in mouse ependyma. Fractional contribution of each transgene-associated barcode in mouse ependyma RNA and AAV4 viral library input following injection at low (5E10 vg), medium (1E11 vg), and high (5E11 vg) vector doses.

FIG. 5. hEpendyma promoter library use in mouse ependyma. Quantification of read enrichment in RNA output relative to the viral library input. Marked enrichment was exhibited by a positive control, the ubiquitous iCAG promoter and by the hVWA3a promoter.

FIG. 6. Fractional contribution of hEpendyma promoters in Rhesus ependyma-containing samples—eGFP. The same library used in FIG. 4 was prepared as AAV2 and injected into the lateral ventricle of two adult rhesus macaques at 2E13 vg total. At 3 weeks post-injection, regions at the ventricle margins, including down through the spinal cord, were micro-dissected. RNA was isolated, converted to cDNA and PCR products containing the 3-letter barcode were subjected to amplicon sequencing. Colored bars indicate the relative contribution of each transgene.

FIG. 7. hEpendyma library round 2—Introduce promoter intron to increase expression. Original transgenes were modified to express human apolipoprotein 2 (ApoE2) cDNA and include a short (133 bp) and long (951 bp) intron within the promoter region to increase expression by intron-mediated enhancement. Short flanking sequences known to promote efficient splicing were also included (blue and green bars).

FIG. 8. hEpendyma promoter library-derived mRNA splices correctly and encodes ApoE protein. To test splicing efficiency in the intron-containing variants, plasmids were transfected into HEK293 cells. Correct splicing was validated by amplifying across the intron-containing region in cDNA (C) vs plasmid DNA (D).

FIG. 9. hEpendyma promoter library-derived mRNAs splice correctly and encode ApoE protein. Western blot measuring ApoE protein output from no intron (N) and intron-containing (Short S, Long L) variants in HEK293 cell lysates and culture media.

FIG. 10. Intron-containing promoter used in RhEpendyma. A library of six different promoters containing no, short, or long introns were prepared in a single AAV2 and injected into the lateral ventricle of two adult rhesus macaques with a total dose of 2.8e13 vg. After four weeks, the ventricle margins were micro-dissected. As before, amplicon sequencing of a product containing a unique 3-letter barcode in the 3′ UTR was used to assess relative promoter use in vivo. All hVWA3a variants showed relative enrichment in vivo over the input library.

FIG. 11. hEpendyma promoter drives expression in mice. Adult APOE−/− (null) mice were injected with serotype AAV4 delivering APOE2 under the hVWA3a promoter to their right lateral ventricle. Ependyma tissue was microdissected and protein extracted for APOE2 quantification by automated Western blot technology (WES) compared to uninjected brain tissue.

FIG. 12. hEpendyma promoter drives higher expression than unbiquitous CAG promoter in mice. APOE−/− (null) mice were injected with serotype AAV4 delivering APOE2 under either a ubiquitous CAG promoter or the hVWA3a promoter to their right lateral ventricle at equal doses. Protein was extracted from ependymal tissues microdissected from all animals and subjected to automated Western blot technology (WES). From the intensity of the bands, APOE2 driven by hVWA3a expressed higher amounts of APOE2 protein than the CAG promoter.

FIG. 13. Peptide modified AAV1 capsid with human ependymal specific promoter: ERDRpAAV1.hVWA3a.eGFP. Positive eGFP fluorescent signal is restricted to the ependymal cells lining the ventricles.

FIG. 14. hVWA3a promoter segment (SEQ ID NO: 1).

FIG. 15. hVWA3a promoter segment with short intron (underline) (133 bp) (SEQ ID NO: 2).

FIG. 16. hVWA3a promoter segment with long intron (underline) (951 bp) (SEQ ID NO: 3).

FIG. 17. Schematic of the AAV transgene that uses upstream regulatory sequence from the human Von Willebrand Factor A Domain 3A (VWA3a) gene to drive ependyma-specific expression of human APOE2. A short β-globin/IgG chimeric intron (133 bp) was inserted downstream the transcription start site to enhance transcription through intron-mediated enhancement and a strong Kozak sequence was included to initiate APOE2 translation. The entire transgene is flanked by AAV2 inverted terminal repeats (ITRs).

FIG. 18. hVWA3a promoter short intron hApoE2 expression construct for AAV (SEQ ID NO: 4).

FIG. 19. pmAAV1.ERDR.hVWA3a.APOE2 @7E10 vg significantly reduces ThioS positive signal in cortex compared to vehicle-treated controls. pmAAV1.ERDR.hVWA3a.APOE2 delivered to Alzheimer's Disease mouse models that are homozygous for human APOE4. There is a significant reduction (p<0.05) in % of ThioS positive cells in mice treated with the high dose of 7E10 vg relative to vehicle treated control mice. Low dose: 7E9 vg; Mid dose: 2E10 vg; High dose: 7E10 vg. ThioS stains the B-pleated sheets found within amyloid plaques in mice (used as a marker of dense core plaques as opposed to diffuse plaques). A difference in ThioS but not oligomeric antibody staining (IBL) would indicate that AAV is preventing formation of dense core plaques rather than plaques formation generally.

FIG. 20. pmAAV1.ERDR.hVWA3a.APOE2 @7E10 vg significantly reduces Amyloid-Beta positive signal in cortex compared to vehicle-treated controls. pmAAV1.ERDR.hVWA3a.APOE2 delivered to Alzheimer's Disease mouse models that are homozygous for human APOE4. There is significant reduction for cells stained for Amyloid-Beta (p<0.05) in mice treated with 7E10 vg relative to vehicle treated mice. Low dose: 7E9 vg; Mid dose: 2E10 vg; High dose: 7E10 vg.

FIG. 21. Viral genome copies assayed by QPCR on DNA against hVWA3a promoter sequence. pmAAV1.ERDR.hVWA3a.APOE2 delivered to Alzheimer's Disease mouse models that are homozygous for human APOE4. DNA lysates from mouse brains treated at all three doses of vector are positive when assayed at the non-transcribed region of the human VWA3a promoter sequence. Control (vehicle) treated AD mice as well as control treated WT mice showed only background levels of hVWA3a that were below detectable range by standard curve. Low dose: 7E9 vg; Mid dose: 2E10 vg; High dose: 7E10 vg.

FIG. 22. Viral genome copies assayed by QPCR on DNA against hVWA3a promoter sequence. Viral genome copies of pmAAV1.ERDR.hVWA3a.APOE2 are detectable in ependymal tissues, cortical tissues, and hippocampal tissues in the brains of nonhuman primates treated with pmAAV1.ERDR.hVWA3a.APOE2 at three different doses. Brain tissue DNA lysates were assayed for total genome copies assayed at the non-transcribed region of the human VWA3a promoter sequence. Naïve samples are from NHPs that did not receive pmAAV1.ERDR.hVWA3a.APOE2. Key top to bottom is same as left to right in graphs.

FIG. 23. Expression of APOE2 in a triple transgenic mouse model of Alzheimer's disease. APOE4XAPP/PS1 are triple transgenic mice expressing a chimeric mouse/human amyloid precursor protein, a mutant human presenilin 1, and human APOE4, creating a model with many aspects of the human condition. Images show cortical amyloid-beta (Aβ; red) with a neuronal stain (DAPI; blue) at 3 months, 4 months, 5 months, and 6 months of age if left untreated. The study paradigm injected mice at 4 months when plaque accumulation started and necropsied at 6 months of age. Mice were injected intraventricularly with pmAAV1.ERDR.hVWA3a.ApoE2 in ascending doses of 7E9, 2E10 and 7E10 vg. Readouts of genome copy expression is shown in FIG. 21.

FIG. 24. APOE2 expression is beneficial for plaque deposition in APOE4XAPP/PS1 mice. ThioS staining on left shows plaques 2 months after delivery of pmAAV1.ERDR.hVWA3a.ApoE2. Quantitative graphs show a significant reduction in ThioS levels and soluble AB42 levels in mice that received the dose of 7E10 vg.

FIG. 25. APOE2 expression reduces plaque density and plaque size in AD mouse model. pmAAV1.ERDR.hVWA3a.ApoE2 at 7E10 vg significantly reduced plaque parameters relative to untreated age matched AD mice.

FIG. 26. Training images for grading parameters for glia. Brain sections were stained for Iba1 (a marker for microglia; blue); GFAP (a marker for astrocytes; green); and amyloid-beta (a marker for plaques; red) and scored for relative levels of glia staining near plaques.

FIG. 27. Virally expressed APOE2 prevents microgliosis near plaques. Two blinded scientists trained with images from FIG. 26 scored microglia (Iba1; blue) near plaques (Aβ; red) in brains from mice dosed with pmAAV1.ERDR.hVWA3a.ApoE2.

FIG. 28. Staining of AD mouse brains at 3, 4, 5, and 6 months of age. Top panel shows cortical images stained with GFAP (green) and Aβ (red). Bottom plane1 shows cortical images stained with Iba1 (blue) Aβ (red).

FIG. 29. Preliminary assessment of AD mice have high variability in microgliosis. AD mice dosed with pmAAV1.ERDR.hVWA3a.ApoE2 show even higher variability in microgliosis.

FIG. 30. Graphical representation of GFAP scoring near plaques. Delivery of APOE2 to AD mice has no significant effect on astrocyte reactivity near plaques as assessed by blind pathological scoring.

FIG. 31. Virally expressed APOE2 prevents synapse loss near plaques. Brain images of AD mice injected with pmAAV1.ERDR.hVWA3a.ApoE2 at 7E10 vg or vehicle control. PSD95 (post-synaptic density −95) staining of synaptic terminals is more apparent in dosed animals.

FIG. 32. Quantitation of synapse integrity of AD mice dosed with APOE2. Synapses proximal (near; red) and distal (far; blue) from plaques were quantified from histological images. Only mice dosed with 7E10 vg of the therapeutic showed similar amounts of synapse densities both near and far from plaques. There was significantly less synaptic density near plaques relative to far from plaques in all other AD treatment groups (graph on left). Graphs on the right compare “near” and “far” synaptic densities between groups revealing that there are significantly higher synaptic densities “near” plaques in the high dose (7E10 vg) group relative to all other groups.

FIGS. 33A-E. Ependymal cell expression of APOE2 driven by a novel AAV capsid and promoter. (FIG. 33A) In Situ hybridization showing human APOE expression in the ependymal cells of the ventricle in a APOE KO mouse. (FIG. 33B) Western blot for APOE showing that ependymally produced AAV derived APOE2 in the cortex of the APOE KO mice is approximately 10% (FIG. 33C) that of endogenous level. (FIG. 33D) Viral genome copies in tissue extracted from each mouse [(F (4, 35)=5.546 p=0.0014) Post Hoc Tukey's multiple comparisons test]. (FIG. 33E) qRTPCR for human APOE normalized to vehicle treated animals (F (4, 26)=2.890 p=0.0419 Post Hoc Dunnett's test to vehicle). (E) Line graph showing a significant correlation (p=0.0445) between mRNA for APOE and viral genome copies. N indicated as each mouse is an individual dot. * p<0.05, ** p<0.01, ***p<0.001.

FIGS. 34A-E. POE2 reduces plaque deposition, number, and size in a dose-dependent manner. (FIG. 34A) IHC for thioS in the cortex of dosed APP/PS1/APOE4 animals. (FIG. 34B) Percent cortex coverage by ThioS staining is significantly lower in the high dose animals (F (3, 27)=4.310 p=0.0329). (FIG. 34C) The percent cortical coverage by ThioS is significantly correlated (p=0.0112) to the number of viral genome copies in the brain sample from each mouse. (FIG. 34D) Plaque number (F (3, 27)=3.597 p=0.0263) and (FIG. 34E) plaque size (F (3, 27)=4.113 p=0.0159) both show a significant effect in the high dose group. n indicated as each mouse is an individual dot with open circles as females and closed as males. Post Hoc tests are shown as Dunnett's multiple comparisons test comparing with vehicle. p*p<0.05, ** p<0.01.

FIGS. 35A-D. APOE2 reduces microgliosis near plaques. (FIG. 35A) IHC for IBA1 and o Aβ in the cortex of dosed APP/PS1/APOE4 animals. (FIG. 35B) average microglial response score (F (3, 26)=4.529 p=0.0110) shows a reduction in microglial activation in the high and mid dose groups and (FIG. 35C) is significantly correlated (p=0.0081) to the number of viral genome copies in that mouse. (FIG. 35D) This reduction is due to a decrease in the number of plaques scored as a 4 and an increase in the number of plaques scored a 1. n indicated as each mouse is an individual dot with open circles as females and closed as males. Post Hoc tests are shown as Dunnett's multiple comparisons test comparing with vehicle. p*p<0.05.

FIGS. 36A-D. APOE2 reduces synaptic loss near. (FIG. 36A) IHC for PSD95 and oAβ in the cortex of dosed APP/PS1/APOE4 animals. (FIG. 36B) Synapse density is unchanged far from plaques but (FIG. 36C) significantly increased near plaques (F (3, 27)=5.153 p=0.0060). (FIG. 36D) this leads to a significant decrease in the percent synapse loss in the high dose animals compared with vehicle (F (3, 27)=3.693 p=0.0239). n indicated as each mouse is an individual dot with open circles as females and closed as males. Post Hoc tests are shown as Dunnett's multiple comparisons test comparing with vehicle. p*p<0.05.

FIGS. 37A-D. Effect of APOE2 on oliogmeric Aβ. (FIG. 37A) Percent cortex coverage by Aβ staining is significantly lower in the high dose animals (F (3, 27)=6.336, p=0.0022). (FIG. 37B) The percent cortical coverage by ThioS is significantly correlated (p=0.0173) to the number of viral genome copies in that mouse. (C FIG. 37) ELISA shows that SDS soluble (F (3, 28)=3.497 p=0.0285) and (FIG. 37D) Formic acid soluble (F (3, 30)=3.741 p=0.0214) Aβ both show a significant effect in the high dose group. n indicated as each mouse is an individual dot with open circles as females and closed as males. Post Hoc tests are shown as Dunnett's multiple comparisons test comparing with vehicle. p*p<0.05, ** p<0.01.

FIGS. 38A-E. APOE2 does not affect astrocyte reactivity near plaques. (FIG. 38A) Representative images of the four-point scale used to assess micro and astroglia reactivity to plaques. (FIG. 38B) IHC for GFAP and oAβ in the cortex of dosed APP/PS1/APOE4 animals. (FIG. 38C) Average astrocyte response score (F (3, 26)=1.634 p=0.2057) shows no change between groups and (FIG. 38D) is not significantly (p=0.1904) correlated to viral genome number. (FIG. 38E) There is no difference between groups in the number of plaques scored into each category. n indicated as each mouse is an individual dot with open circles as females and closed as males. Post Hoc tests are shown as Dunnett's multiple comparisons test comparing with vehicle. p*p<0.05.

FIGS. 39A-C. APOE2 does not affect neuritic dystrophies. (FIG. 39A) IHC for Smi-312 and oAβ in the cortex of dosed APP/PS1/APOE4 animals. (FIG. 39B) Analysis shows no difference in the number of dystrophies counted per plaque (F (3, 25)=0.4710; p=0.7052) even (FIG. 39C) when accounting for plaque size (F (3, 25)=1.127 p=0.3569).

FIG. 40. hEpendyma promoter drives ependyma-localized APOE transcription in NHP following ICV delivery. A total of 1E13 vg of pmAAV1.ERDR.hVWA3a.ApoE2 vector were injected unilaterally into the lateral ventricle of an adult African green monkey. Tissues were harvested for sectioning at 60 days post-injection and transgene expression was monitored by RNA fluorescent in situ hybridization (RNA-FISH). Probes designed to target human APOE exhibit strong overlap with endogenous African Green APOE due to high sequence homology. To specify transcript origin, we relied on location. Endogenous APOE transcription occurs only in astrocytes and microglia, therefore we can attribute ependyma-localized signal, defined by overlap with the ependyma-specific gene FoxJ1 (outlined with white dashed lines), as transgene-derived APOE2. Hoechst H33258 identifies tissue DNA.

FIG. 41. hEpendyma promoter increases CSF APOE in NHP following ICV delivery. A total of 1E13 vg of pmAAV1.ERDR.hVWA3a.ApoE2 vector were injected unilaterally into the lateral ventricle of an adult African Green monkey. CSF was collected at baseline, 30-, 45-, and 60-days post-injection and APOE protein was measured by automated western blot. All values were normalized to baseline.

### DETAILED DESCRIPTION

Here, the inventors sought to identify promoter sequences capable of driving ependyma-specific expression in mouse and human brain which could in turn be used in gene therapy modalities to drive expression of secreted proteins to treat neurological disease. The inventors chose to first identify genes, and then by proxy the related promoters, that are insensitive to neurological disease state and age. Such promoters could be used to drive transgene expression in the ependyma, the layer of epithelial cells lining the ventricles of the brain. Following infection of these cells with AAV, secreted proteins can enter the ventricles and distribute throughout the entire brain by way of the cerebrospinal fluid. And by eliminating promoters that might be negatively impacted by the altered gene expression patterns in disease tissues, stronger transgene expression could be achieved.

To accomplish this, the inventors obtained ependyma and adjacent ependyma-free samples from normal and diseased brain (Alzheimer's disease, Huntington's disease, Frontotemporal dementia, etc,). Using RNA sequencing, the inventors identified genes whose expression was enriched in ependyma-containing samples and maintained regardless of disease state. Further evidence for their specificity was validated using published datasets, including the Allen Brain Institute in situ hybridization library. As promoters are loosely defined structures, they isolated genomic sequences (˜1100-2500 bp) upstream the transcription start site from top gene candidates (11 promoters) and placed them upstream a GFP reporter and unique 3 letter RNA barcode within an AAV-compatible transgene (flanked by ITRs). Plasmids containing the different promoters were pooled, prepared as AAV4 or AAV2, and injected directly into the ventricles of mouse or rhesus macaque, respectively. Ependyma-containing tissues were micro-dissected and amplicon sequencing was performed on the region surrounding the 3-letter barcode. Output was compared to library input to determine enrichment. Inclusion of upstream introns has been shown to increase tissue expression. The top hits (6 promoters) identified in the first screen were further modified to include a short (133 bp) or long (951 bp) intron. Individual versions were again identified by a unique 3 letter barcode in the 3′UTR. Transgenes that included variants of the human Von Willebrand factor A domain containing 3a (hVWA3a) were most highly enriched in the final screen and chosen for further studies.

These and other aspects of the disclosure are discussed in greater detail below.

### I. ADENO-ASSOCIATED VIRUS (AAV) VECTORS

Adeno-associated virus (AAV) is a small nonpathogenic virus of the parvoviridae family. To date, numerous serologically distinct AAVs have been identified, and more than a dozen have been isolated from humans or primates. AAV is distinct from other members of this family by its dependence upon a helper virus for replication.

AAV genomes can exist in an extrachromosomal state without integrating into host cellular genomes; possess a broad host range; transduce both dividing and non-dividing cells in vitro and in vivo and maintain high levels of expression of the transduced genes. AAV viral particles are heat stable; resistant to solvents, detergents, changes in pH, and temperature; and can be column purified and/or concentrated on CsClgradients or by other means. The AAV genome comprises a single-stranded deoxyribonucleic acid (ssDNA), either positive- or negative-sensed. The approximately 4.7 kb genome of AAV consists of one segment of single stranded DNA of either plus or minus polarity. The ends of the genome are short-inverted terminal repeats (ITRs) that can fold into hairpin structures and serve as the origin of viral DNA replication.

An AAV “genome” refers to a recombinant nucleic acid sequence that is ultimately packaged or encapsulated to form an AAV particle. An AAV particle often comprises an AAV genome packaged with AAV capsid proteins. In cases where recombinant plasmids are used to construct or manufacture recombinant vectors, the AAV vector genome does not include the portion of the “plasmid” that does not correspond to the vector genome sequence of the recombinant plasmid. This non-vector genome portion of the recombinant plasmid is referred to as the “plasmid backbone,” which is important for cloning and amplification of the plasmid, a process that is needed for plasmid propagation and production, but is not itself packaged or encapsulated into viral particles. Thus, an AAV vector “genome” refers to nucleic acid that is packaged or encapsulated by AAV capsid proteins.

The AAV virion (particle) is a non-enveloped, icosahedral particle approximately 25 nm in diameter that comprises an AAV capsid. The AAV particle comprises an icosahedral symmetry comprised of three related capsid proteins, VP1, VP2 and VP3, which interact together to form the capsid. The genome of most native AAVs often contain two open reading frames (ORFs), sometimes referred to as a left ORF and a right ORF. The right ORF often encodes the capsid proteins VP1, VP2, and VP3. These proteins are often found in a ratio of 1:1:10 respectively, but may be in varied ratios, and are all derived from the right-hand ORF. The VP1, VP2 and VP3 capsid proteins differ from each other by the use of alternative splicing and an unusual start codon. Deletion analysis has shown that removal or alteration of VP1 which is translated from an alternatively spliced message results in areduced yield of infectious particles. Mutations within the VP3 coding region result in the failure to produce any single-stranded progeny DNA or infectious particles. In certain embodiments, the genome of an AAV particle encodes one, two or all three VP1, VP2 and VP3 polypeptides.

The left ORF often encodes the non-structural Rep proteins, Rep 40, Rep 52, Rep 68 and Rep 78, which are involved in regulation of replication and transcription in addition to the production of single-stranded progeny genomes. Two of the Rep proteins have been associated with the preferential integration of AAV genomes into a region of the q arm of human chromosome 19. Rep68/78 have been shown to possess NTP binding activity as well as DNA and RNA helicase activities. Some Rep proteins possess a nuclear localization signal as well as several potential phosphorylation sites. In certain embodiments the genome of an AAV (e.g., an rAAV) encodes some or all of the Rep proteins. In certain embodiments the genome of an AAV (e.g., an rAAV) does not encode the Rep proteins. In certain embodiments one or more of the Rep proteins can be delivered in trans and are therefore not included in an AAV particle comprising a nucleic acid encoding a polypeptide.

The ends of the AAV genome comprise short-inverted terminal repeats (ITR) which have the potential to fold into T-shaped hairpin structures that serve as the origin of viral DNA replication. Accordingly, the genome of an AAV comprises one or more (e.g., a pair of) ITR sequences that flank a single stranded viral DNA genome. The ITR sequences often have a length of about 145 bases each. Within the ITR region, two elements have been described which are believed to be central to the function of the ITR, a GAGC repeat motif and the terminal resolution site (trs). The repeat motif has been shown to bind Rep when the ITR is in either a linear or hairpin conformation. This binding is thought to position Rep68/78 for cleavage at the trs which occurs in a site- and strand-specific manner. In addition to their role in replication, these two elements appear to be central to viral integration. Contained within the chromosome 19 integration locus is a Rep binding site with an adjacent trs. These elements have been shown to be functional and necessary for locus specific integration.

The term “recombinant,” as a modifier of vector, such as recombinant viral, e.g., lentivirus or parvovirus (e.g., AAV) vectors, as well as a modifier of sequences such as recombinant nucleic acid sequences and polypeptides, means that the compositions have been manipulated (i.e., engineered) in a fashion that generally does not occur in nature. A particular example of a recombinant vector, such as an AAV, retroviral, or lentiviral vector would be where a nucleic acid sequence that is not normally present in the wild-type viral genome is inserted within the viral genome. An example of a recombinant nucleic acid sequence would be where a nucleic acid (e.g., gene) encodes an inhibitory RNA cloned into a vector, with or without 5′, 3′ and/or intron regions that the gene is normally associated within the viral genome. Although the term “recombinant” is not always used herein in reference to vectors, such as viral vectors, as well as sequences such as polynucleotides, “recombinant” forms including nucleic acid sequences, polynucleotides, transgenes, etc. are expressly included in spite of any such omission.

A recombinant viral “vector” is derived from the wild-type genome of a virus by using molecular methods to remove part of the wild type genome from the virus, and replacing with a non-native nucleic acid, such as a nucleic acid sequence. Typically, for example, for AAV, one or both inverted terminal repeat (ITR) sequences of the AAV genome are retained in the recombinant AAV vector. A “recombinant” viral vector (e.g., rAAV) is distinguished from a viral (e.g., AAV) genome, since part of the viral genome has been replaced with a non-native sequence with respect to the viral genomic nucleic acid such a nucleic acid encoding a transactivator or nucleic acid encoding an inhibitory RNA or nucleic acid encoding a therapeutic protein. Incorporation of such non-native nucleic acid sequences therefore defines the viral vector as a “recombinant” vector, which in the case of AAV can be referred to as a “rAAV vector.”

In certain embodiments, an AAV (e.g., a rAAV) comprises two ITRs. In certain embodiments, an AAV (e.g., a rAAV) comprises a pair of ITRs. In certain embodiments, an AAV (e.g., a rAAV) comprises a pair of ITRs that flank (i.e., are at each 5′ and 3′ end) of a nucleic acid sequence that at least encodes a polypeptide having function or activity.

An AAV vector (e.g., rAAV vector) can be packaged and is referred to herein as an “AAV particle” for subsequent infection (transduction) of a cell, ex vivo, in vitro or in vivo. Where a recombinant AAV vector is encapsulated or packaged into an AAV particle, the particle can also be referred to as a “rAAV particle.” In certain embodiments, an AAV particle is a rAAV particle. A rAAV particle often comprises a rAAV vector, or a portion thereof. A rAAV particle can be one or more rAAV particles (e.g., a plurality of AAV particles). rAAV particles typically comprise proteins that encapsulate or package the rAAV vector genome (e.g., capsid proteins). It is noted that reference to a rAAV vector can also be used to reference a rAAV particle.

Any suitable AAV particle (e.g., rAAV particle) can be used for a method or use herein. A rAAV particle, and/or genome comprised therein, can be derived from any suitable serotype or strain of AAV. A rAAV particle, and/or genome comprised therein, can be derived from two or more serotypes or strains of AAV. Accordingly, a rAAV can comprise proteins and/or nucleic acids, or portions thereof, of any serotype or strain of AAV, wherein the AAV particle is suitable for infection and/or transduction of a mammalian cell. Non-limiting examples of AAV serotypes include AAV1, AAV2, AAV3, AAV4, AAV5, AAV6, AAV7, AAV8, AAV9, AAV10, AAV11, AAV12, AAV-rh74, AAV-rh10 and AAV-2i8.

In certain embodiments a plurality of rAAV particles comprises particles of, or derived from, the same strain or serotype (or subgroup or variant). In certain embodiments a plurality of rAAV particles comprise a mixture of two or more different rAAV particles (e.g., of different serotypes and/or strains).

As used herein, the term “serotype” is a distinction used to refer to an AAV having a capsid that is serologically distinct from other AAV serotypes. Serologic distinctiveness is determined on the basis of the lack of cross-reactivity between antibodies to one AAV as compared to another AAV. Such cross-reactivity differences are usually due to differences in capsid protein sequences/antigenic determinants (e.g., due to VP1, VP2, and/or VP3 sequence differences of AAV serotypes). Despite the possibility that AAV variants including capsid variants may not be serologically distinct from a reference AAV or other AAV serotype, they differ by at least one nucleotide or amino acid residue compared to the reference or other AAV serotype.

In certain embodiments, a rAAV vector based upon a first serotype genome corresponds to the serotype of one or more of the capsid proteins that package the vector. For example, the serotype of one or more AAV nucleic acids (e.g., ITRs) that comprises the AAV vector genome corresponds to the serotype of a capsid that comprises the rAAV particle.

In certain embodiments, a rAAV vector genome can be based upon an AAV (e.g., AAV2) serotype genome distinct from the serotype of one or more of the AAV capsid proteins that package the vector. For example, a rAAV vector genome can comprise AAV2 derived nucleic acids (e.g., ITRs), whereas at least one or more of the three capsid proteins are derived from a different serotype, e.g., an AAV1, AAV3, AAV4, AAV5, AAV6, AAV7, AAV8, AAV9, AAV10, AAV11, AAV12, Rh10, Rh74 or AAV-2i8 serotype or variant thereof.

In certain embodiments, a rAAV particle or a vector genome thereof related to a reference serotype has a polynucleotide, polypeptide or subsequence thereof that comprises or consists of a sequence at least 60% or more (e.g., 65%, 70%, 75%, 80%, 85%, 90%, 95%, 96%, 97%, 98%, 99%, 99.1%, 99.2%, 99.3%, 99.4%, 99.5%, etc.) identical to a polynucleotide, polypeptide or subsequence of an AAV1, AAV2, AAV3, AAV4, AAV5, AAV6, AAV7, AAV8, AAV9, AAV10, AAV11, AAV12, Rh10, Rh74 or AAV-2i8 particle. In particular embodiments, a rAAV particle or a vector genome thereof related to a reference serotype has a capsid or ITR sequence that comprises or consists of a sequence at least 60% or more (e.g., 65%, 70%, 75%, 80%, 85%, 90%, 95%, 96%, 97%, 98%, 99%, 99.1%, 99.2%, 99.3%, 99.4%, 99.5%, etc.) identical to a capsid or ITR sequence of an AAV1, AAV2, AAV3, AAV4, AAV5, AAV6, AAV7, AAV8, AAV9, AAV10, AAV11, AAV12, Rh10, Rh74 or AAV-2i8 serotype.

In certain embodiments, a method herein comprises use, administration or delivery of an rAAV1, rAAV2, rAAV3, rAAV4, rAAV5, rAAV6, rAAV7, rAAV8, rAAV9, rAAV10, rAAV11, rAAV12, rRh10, rRh74 or rAAV-2i8 particle.

In certain embodiments, a method herein comprises use, administration or delivery of a rAAV2 particle. In certain embodiments a rAAV2 particle comprises an AAV2 capsid. In certain embodiments a rAAV2 particle comprises one or more capsid proteins (e.g., VP1, VP2 and/or VP3) that are at least 60%, 65%, 70%, 75% or more identical, e.g., 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to a corresponding capsid protein of a native or wild-type AAV2 particle. In certain embodiments a rAAV2 particle comprises VP1, VP2 and VP3 capsid proteins that are at least 75% or more identical, e.g., 80%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to a corresponding capsid protein of a native or wild-type AAV2 particle. In certain embodiments, a rAAV2 particle is a variant of a native or wild-type AAV2 particle. In some aspects, one or more capsid proteins of an AAV2 variant have 1, 2, 3, 4, 5, 5-10, 10-15, 15-20 or more amino acid substitutions compared to capsid protein(s) of a native or wild-type AAV2 particle.

In certain embodiments a rAAV9 particle comprises an AAV9 capsid. In certain embodiments a rAAV9 particle comprises one or more capsid proteins (e.g., VP1, VP2 and/or VP3) that are at least 60%, 65%, 70%, 75% or more identical, e.g., 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to a corresponding capsid protein of a native or wild-type AAV9 particle. In certain embodiments a rAAV9 particle comprises VP1, VP2 and VP3 capsid proteins that are at least 75% or more identical, e.g., 80%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to a corresponding capsid protein of a native or wild-type AAV9 particle. In certain embodiments, a rAAV9 particle is a variant of a native or wild-type AAV9 particle. In some aspects, one or more capsid proteins of an AAV9 variant have 1, 2, 3, 4, 5, 5-10, 10-15, 15-20 or more amino acid substitutions compared to capsid protein(s) of a native or wild-type AAV9 particle.

In certain embodiments, a rAAV particle comprises one or two ITRs (e.g., a pair of ITRs) that are at least 75% or more identical, e.g., 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to corresponding ITRs of a native or wild-type AAV1, AAV2, AAV3, AAV4, AAVS, AAV6, AAV7, AAV8, AAV9, AAV10, AAV11, AAV12, AAV-rh74, AAV-rh10 or AAV-2i8, as long as they retain one or more desired ITR functions (e.g., ability to form a hairpin, which allows DNA replication; integration of the AAV DNA into a host cell genome; and/or packaging, if desired).

In certain embodiments, a rAAV2 particle comprises one or two ITRs (e.g., a pair of ITRs) that are at least 75% or more identical, e.g., 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to corresponding ITRs of a native or wild-type AAV2 particle, as long as they retain one or more desired ITR functions (e.g., ability to form a hairpin, which allows DNA replication; integration of the AAV DNA into a host cell genome; and/or packaging, if desired).

In certain embodiments, a rAAV9 particle comprises one or two ITRs (e.g., a pair of ITRs) that are at least 75% or more identical, e.g., 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to corresponding ITRs of a native or wild-type AAV2 particle, as long as they retain one or more desired ITR functions (e.g., ability to form a hairpin, which allows DNA replication; integration of the AAV DNA into a host cell genome; and/or packaging, if desired).

A rAAV particle can comprise an ITR having any suitable number of “GAGC” repeats. In certain embodiments an ITR of an AAV2 particle comprises 1, 2, 3, 4, 5, 6, 7, 8, 9 or 10 or more “GAGC” repeats. In certain embodiments a rAAV2 particle comprises an ITR comprising three “GAGC” repeats. In certain embodiments a rAAV2 particle comprises an ITR which has less than four “GAGC” repeats. In certain embodiments a rAAV2 particle comprises an ITR which has more than four “GAGC” repeats. In certain embodiments an ITR of a rAAV2 particle comprises a Rep binding site wherein the fourth nucleotide in the first two “GAGC” repeats is a C rather than a T.

Exemplary suitable length of DNA can be incorporated in rAAV vectors for packaging/encapsidation into a rAAV particle can about 5 kilobases (kb) or less. In particular, embodiments, length of DNA is less than about 5 kb, less than about 4.5 kb, less than about 4 kb, less than about 3.5 kb, less than about 3 kb, or less than about 2.5 kb.

rAAV vectors that include a nucleic acid sequence that directs the expression of an RNAi or polypeptide can be generated using suitable recombinant techniques known in the art (e.g., see Sambrook et al., 1989). Recombinant AAV vectors are typically packaged into transduction competent AAV particles and propagated using an AAV viral packaging system. A transduction competent AAV particle is capable of binding to and entering a mammalian cell and subsequently delivering a nucleic acid cargo (e.g., a heterologous gene) to the nucleus of the cell. Thus, an intact rAAV particle that is transduction-competent is configured to transduce a mammalian cell. A rAAV particle configured to transduce a mammalian cell is often not replication competent and requires additional protein machinery to self-replicate. Thus, a rAAV particle that is configured to transduce a mammalian cell is engineered to bind and enter a mammalian cell and deliver a nucleic acid to the cell, wherein the nucleic acid for delivery is often positioned between a pair of AAV ITRs in the rAAV genome.

Suitable host cells for producing transduction-competent AAV particles include but are not limited to microorganisms, yeast cells, insect cells, and mammalian cells that can be, or have been, used as recipients of a heterologous rAAV vectors. Cells from the stable human cell line, HEK293 (readily available through, e.g., the American Type Culture Collection under Accession Number ATCC CRL1573) can be used. In certain embodiments a modified human embryonic kidney cell line (e.g., HEK293), which is transformed with adenovirus type-5 DNA fragments, and expresses the adenoviral E1a and E1b genes is used to generate recombinant AAV particles. The modified HEK293 cell line is readily transfected, and provides a particularly convenient platform in which to produce rAAV particles. Methods of generating high titer AAV particles capable of transducing mammalian cells are known in the art. For example, AAV particles can be made as set forth in Wright, 2008 and Wright, 2009.

In certain embodiments, AAV helper functions are introduced into the host cell by transfecting the host cell with an AAV helper construct either prior to, or concurrently with, the transfection of an AAV expression vector. AAV helper constructs are thus sometimes used to provide at least transient expression of AAV rep and/or cap genes to complement missing AAV functions necessary for productive AAV transduction. AAV helper constructs often lack AAV ITRs and can neither replicate nor package themselves. These constructs can be in the form of a plasmid, phage, transposon, cosmid, virus, or virion. A number of AAV helper constructs have been described, such as the commonly used plasmids pAAV/Ad and pIM29+45 which encode both Rep and Cap expression products. A number of other vectors are known which encode Rep and/or Cap expression products.

An “expression vector” is a specialized vector that contains a gene or nucleic acid sequence with the necessary regulatory regions needed for expression in a host cell. An expression vector may contain at least an origin of replication for propagation in a cell and optionally additional elements, such as a heterologous nucleic acid sequence, expression control element (e.g., a promoter, enhancer), intron, ITR(s), and polyadenylation signal.

### II. THERAPEUTIC AGENTS

In some embodiments, viral gene transfer methods can be used to introduce nucleic acids in mammalian cells or target tissues. Such methods can be used to administer nucleic acids encoding inhibitory RNAs, non-coding RNAs, and/or therapeutic proteins to cells in culture or in a host organism.

### A. Inhibitory RNAs

“RNA interference (RNAi)” is the process of sequence-specific, post-transcriptional gene silencing initiated by siRNA. During RNAi, siRNA induces degradation of target mRNA with consequent sequence-specific inhibition of gene expression.

An “inhibitory RNA,” “RNAi,” “small interfering RNA” or “short interfering RNA” or “siRNA” molecule, “short hairpin RNA” or “shRNA” molecule, or “miRNA” is an RNA duplex of nucleotides that is targeted to a nucleic acid sequence of interest. As used herein, the term “siRNA” is a generic term that encompasses the subset of shRNAs and miRNAs. An “RNA duplex” refers to the structure formed by the complementary pairing between two regions of an RNA molecule. siRNA is “targeted” to a gene in that the nucleotide sequence of the duplex portion of the siRNA is complementary to a nucleotide sequence of the targeted gene. In certain embodiments, the siRNAs are targeted to the sequence encoding huntingtin. In some embodiments, the length of the duplex of siRNAs is less than 30 base pairs. In some embodiments, the duplex can be 29, 28, 27, 26, 25, 24, 23, 22, 21, 20, 19, 18, 17, 16, 15, 14, 13, 12, 11 or 10 base pairs in length. In some embodiments, the length of the duplex is 19 to 25 base pairs in length. In certain embodiment, the length of the duplex is 19 or 21 base pairs in length. The RNA duplex portion of the siRNA can be part of a hairpin structure. In addition to the duplex portion, the hairpin structure may contain a loop portion positioned between the two sequences that form the duplex. The loop can vary in length. In some embodiments the loop is 5, 6, 7, 8, 9, 10, 11, 12, 13, 14, 15, 16, 17, 18, 19, 20, 21, 22, 23, 24 or 25 nucleotides in length. In certain embodiments, the loop is 18 nucleotides in length. The hairpin structure can also contain 3′ and/or 5′ overhang portions. In some embodiments, the overhang is a 3′ and/or a 5′ overhang 0, 1, 2, 3, 4 or 5 nucleotides in length.

shRNAs are comprised of stem-loop structures which are designed to contain a 5′ flanking region, siRNA region segments, a loop region, a 3′ siRNA region and a 3′ flanking region. Most RNAi expression strategies have utilized short-hairpin RNAs (shRNAs) driven by strong polIII-based promoters. Many shRNAs have demonstrated effective knock down of the target sequences in vitro as well as in vivo, however, some shRNAs which demonstrated effective knock down of the target gene were also found to have toxicity in vivo.

miRNAs are small cellular RNAs (˜22 nt) that are processed from precursor stem loop transcripts. Known miRNA stem loops can be modified to contain RNAi sequences specific for genes of interest. miRNA molecules can be preferable over shRNA molecules because miRNAs are endogenously expressed. Therefore, miRNA molecules are unlikely to induce dsRNA-responsive interferon pathways, they are processed more efficiently than shRNAs, and they have been shown to silence 80% more effectively.

A recently discovered alternative approach is the use of artificial miRNAs (pri-miRNA scaffolds shuttling siRNA sequences) as RNAi vectors. Artificial miRNAs more naturally resemble endogenous RNAi substrates and are more amenable to Pol-II transcription (e.g., allowing tissue-specific expression of RNAi) and polycistronic strategies (e.g., allowing delivery of multiple siRNA sequences). See U.S. Pat. No. 10,093,927, which is incorporated by reference.

The transcriptional unit of a “shRNA” is comprised of sense and antisense sequences connected by a loop of unpaired nucleotides. shRNAs are exported from the nucleus by Exportin-5, and once in the cytoplasm, are processed by Dicer to generate functional siRNAs. “miRNAs” stem-loops are comprised of sense and antisense sequences connected by a loop of unpaired nucleotides typically expressed as part of larger primary transcripts (pri-miRNAs), which are excised by the Drosha-DGCR8 complex generating intermediates known as pre-miRNAs, which are subsequently exported from the nucleus by Exportin-5, and once in the cytoplasm, are processed by Dicer to generate functional siRNAs. “Artificial miRNA” or an “artificial miRNA shuttle vector”, as used herein interchangeably, refers to a primary miRNA transcript that has had a region of the duplex stem loop (at least about 9-20 nucleotides) which is excised via Drosha and Dicer processing replaced with the siRNA sequences for the target gene while retaining the structural elements within the stem loop necessary for effective Drosha processing. The term “artificial” arises from the fact the flanking sequences (˜35 nucleotides upstream and ˜40 nucleotides downstream) arise from restriction enzyme sites within the multiple cloning site of the siRNA. As used herein the term “miRNA” encompasses both the naturally occurring miRNA sequences as well as artificially generated miRNA shuttle vectors.

The siRNA can be encoded by a nucleic acid sequence, and the nucleic acid sequence can also include a promoter. The nucleic acid sequence can also include a polyadenylation signal. In some embodiments, the polyadenylation signal is a synthetic minimal polyadenylation signal or a sequence of six Ts.

In designing RNAi there are several factors that need to be considered, such as the nature of the siRNA, the durability of the silencing effect, and the choice of delivery system. To produce an RNAi effect, the siRNA that is introduced into the organism will typically contain exonic sequences. Furthermore, the RNAi process is homology dependent, so the sequences must be carefully selected so as to maximize gene specificity, while minimizing the possibility of cross-interference between homologous, but not gene-specific sequences. Preferably the siRNA exhibits greater than 80%, 85%, 90%, 95%, 98%, or even 100% identity between the sequence of the siRNA and the gene to be inhibited. Sequences less than about 80% identical to the target gene are substantially less effective. Thus, the greater homology between the siRNA and the gene to be inhibited, the less likely expression of unrelated genes will be affected.

In addition, the size of the siRNA is an important consideration. In some embodiments, the present disclosure relates to siRNA molecules that include at least about 19-25 nucleotides and are able to modulate gene expression. In the context of the present disclosure, the siRNA is preferably less than 500, 200, 100, 50, or 25 nucleotides in length. More preferably, the siRNA is from about 19 nucleotides to about 25 nucleotides in length.

A siRNA target generally means a polynucleotide comprising a region that encodes a polypeptide, or a polynucleotide region that regulates replication, transcription, or translation or other processes important to expression of the polypeptide, or a polynucleotide comprising both a region that encodes a polypeptide and a region operably linked thereto that regulates expression. Any gene being expressed in a cell can be targeted. Preferably, a target gene is one involved in or associated with the progression of cellular activities important to disease or of particular interest as a research object.

### B. Non-Coding RNAs

As evidenced by cDNA cloning projects and genomic tiling arrays, more than 90% of the human genome undergoes transcription but does not code for proteins. These transcriptional products are referred to as non-protein coding RNAs (ncRNAs). A variety of ncRNA transcripts, such as ribosomal RNAs, transfer RNAs, competing endogenous RNA (ceRNA), small nuclear RNA (snRNA), and small nucleolar RNA (snoRNA), are essential for cell function. Similarly, a large number of short ncRNAs such as micro-RNAs (miRNAs), endogenous short interfering RNAs (siRNAs), PIWI-interacting RNAs (piRNAs), and small nucleolar RNAs (snoRNAs) are also known to play important regulatory roles in eukaryotic cells. Recent studies have demonstrated a group of long ncRNA (lncRNA) transcripts that exhibit cell type-specific expression and localize into specific subcellular compartments. lncRNAs are also known to play important roles during cellular development and differentiation supporting the view that they have been selected during the evolutionary process.

LncRNAs appear to have many different functions. In many cases, they seem to play a role in regulating the activity or localization of proteins or serve as organizational frameworks for subcellular structures. In other cases, lncRNAs are processed to yield multiple small RNAs or they may modulate how other RNAs are processed. The latest edition of data produced by the public research consortium GenCode (version #27) catalogs just under 16,000 lncRNAs in the human genome, producing nearly 28,000 transcripts; when other databases are included, more than 40,000 lncRNAs are known.

Interestingly, lncRNAs can influence the expression of specific target proteins at specific genomic loci, modulate the activity of protein binding partners, direct chromatin-modifying complexes to their sites of action, and are post-transcriptionally processed to produce numerous 5′-capped small RNAs. Epigenetic pathways can also regulate the differential expression of lncRNAs.

A growing body of evidence also suggests that aberrantly expressed lncRNAs play important roles in normal physiological processes as well as multiple disease states. lncRNAs are misregulated in various diseases, including ischaemia, heart disease, Alzheimer's disease, psoriasis, and spinocerebellar ataxia type 8. This misregulation has also been shown in various types of cancers, such as breast cancer, colon cancer, prostate cancer, hepatocellular carcinoma and leukemia. Several lncRNAs, e.g., gadd74 and lncRNA-RoR5, modulate cell cycle regulators such as cyclins, cyclin-dependent kinases (CDKs), CDK inhibitors and p53 and thus provide an additional layer of flexibility and robustness to cell cycle progression. In addition, some lncRNAs are linked to mitotic processes such as centromeric satellite RNA, which is essential for kinetochore formation and thus crucial for chromosome segregation during mitosis in humans and flies. Another nuclear lncRNA, MA-lincl, regulates M phase exit by functioning in cis to repress the expression of its neighbouring gene Pura, a regulator of cell proliferation.

lncRNAs are a group that is commonly defined as transcripts of more than 200 nucleotides (e.g., about 200 to about 1200 nt, about 2500 nt, or more) that lack an extended open reading frame (ORF). The term “non-coding RNA” (ncRNA) includes lncRNA as well as shorter transcripts of, e.g., less than about 200 nt, such as about 30 to 200 nt.

Thus, in some embodiments, delivery of a ncRNA, such as to a specific brain structure of interest, corrects aberrant RNA expression levels or modulates levels of disease-causing lncRNA. Accordingly, in some embodiments, the present disclosure provides an rAAV, wherein the viral genome is engineered to encode a therapeutic non-coding RNA (ncRNA). In some embodiments, the ncRNA is a long non-coding RNA (lncRNA) of about 200 nucleotides (nt) in length or greater. In some embodiments, the therapeutic is a ncRNA of about 25 nt or about 30 nt to about 200 nt in length. In some embodiments, the lncRNA is about 200 nt to about 1,200 nt in length. In some embodiments, the JncRNA is about 200 nt to about 1,100, about 1,000, about 900, about 800, about 700, about 600, about 500, about 400, or about 300 nt in length.

### C. CRISPR Systems

Gene editing is a technology that allows for the modification of target genes within living cells. Recently, harnessing the bacterial immune system of CRISPR to perform on demand gene editing revolutionized the way scientists approach genomic editing. The Cas9 protein of the CRISPR system, which is an RNA guided DNA endonuclease, can be engineered to target new sites with relative ease by altering its guide RNA sequence. This discovery has made sequence specific gene editing functionally effective.

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

The CRISPR/Cas nuclease or CRISPR/Cas nuclease system can include a non-coding RNA molecule (guide) RNA, which sequence-specifically binds to DNA, and a Cas protein (e.g., Cas9), with nuclease functionality (e.g., two nuclease domains). One or more elements of a CRISPR system can derive from a type I, type II, or type III CRISPR system, e.g., derived from a particular organism comprising an endogenous CRISPR system, such as Streptococcus pyogenes.

The CRISPR system can induce double stranded breaks (DSBs) at the target site, followed by disruptions as discussed herein. In other embodiments, Cas9 variants, deemed “nickases,” are used to nick a single strand at the target site. Paired nickases can be used, e.g., to improve specificity, each directed by a pair of different gRNAs targeting sequences such that upon introduction of the nicks simultaneously, a 5′ overhang is introduced. In other embodiments, catalytically inactive Cas9 is fused to a heterologous effector domain such as a transcriptional repressor (e.g., KRAB) or activator, to affect gene expression. Alternatively, a CRISPR system with a catalytically inactivated Cas9 further comprises a transcriptional repressor or activator fused to a ribosomal binding protein.

In some aspects, a Cas nuclease and gRNA (including a fusion of crRNA specific for the target sequence and fixed tracrRNA) are introduced into the cell. In general, target sites at the 5′ end of the gRNA target the Cas nuclease to the target site, e.g., the gene, using complementary base pairing. The target site may be selected based on its location immediately 5′ of a protospacer adjacent motif (PAM) sequence, such as typically NGG, or NAG. In this respect, the gRNA is targeted to the desired sequence by modifying the first 20, 19, 18, 17, 16, 15, 14, 14, 12, 11, or 10 nucleotides of the guide RNA to correspond to the target DNA sequence. In general, a CRISPR system is characterized by elements that promote the formation of a CRISPR complex at the site of a target sequence. Typically, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex.

The target sequence may comprise any polynucleotide, such as DNA or RNA polynucleotides. The target sequence may be located in the nucleus or cytoplasm of the cell, such as within an organelle of the cell. Generally, a sequence or template that may be used for recombination into the targeted locus comprising the target sequences is referred to as an “editing template” or “editing polynucleotide” or “editing sequence.” In some aspects, an exogenous template polynucleotide may be referred to as an editing template. In some aspects, the recombination is homologous recombination.

Typically, in the context of an endogenous CRISPR system, formation of the CRISPR complex (comprising the guide sequence hybridized to the target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. The tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of the CRISPR complex, such as by hybridization along at least a portion of the tracr sequence to all or a portion of a tracr mate sequence that is operably linked to the guide sequence. The tracr sequence has sufficient complementarity to a tracr mate sequence to hybridize and participate in formation of the CRISPR complex, such as at least 50%, 60%, 70%, 80%, 90%, 95% or 99% of sequence complementarity along the length of the tracr mate sequence when optimally aligned.

One or more vectors driving expression of one or more elements of the CRISPR system can be introduced into the cell such that expression of the elements of the CRISPR system direct formation of the CRISPR complex at one or more target sites. Components can also be delivered to cells as proteins and/or RNA. For example, a Cas enzyme, a guide sequence linked to a tracr-mate sequence, and a tracr sequence could each be operably linked to separate regulatory elements on separate vectors. The Cas enzyme may be a target gene under the control of a regulated alternative splicing event, as disclosed herein, either as a chimeric target gene minigene or as a target gene for a chimeric minigene transactivator. The gRNA may be under the control of a constitutive promoter.

Alternatively, two or more of the elements expressed from the same or different regulatory elements, may be combined in a single vector, with one or more additional vectors providing any components of the CRISPR system not included in the first vector. The vector may comprise one or more insertion sites, such as a restriction endonuclease recognition sequence (also referred to as a “cloning site”). In some embodiments, one or more insertion sites are located upstream and/or downstream of one or more sequence elements of one or more vectors. When multiple different guide sequences are used, a single expression construct may be used to target CRISPR activity to multiple different, corresponding target sequences within a cell.

A vector may comprise a regulatory element operably linked to an enzyme-coding sequence encoding the CRISPR enzyme, such as a Cas protein. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes are known; for example, the amino acid sequence of S. pyogenes Cas9 protein may be found in the SwissProt database under accession number Q99ZW2.

The CRISPR enzyme can be Cas9 (e.g., from S. pyogenes or S. pneumonia). The CRISPR enzyme can direct cleavage of one or both strands at the location of a target sequence, such as within the target sequence and/or within the complement of the target sequence. The vector can encode a CRISPR enzyme that is mutated with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). In some embodiments, a Cas9 nickase may be used in combination with guide sequence(s), e.g., two guide sequences, which target respectively sense and antisense strands of the DNA target. This combination allows both strands to be nicked and used to induce NHEJ or HDR.

In some embodiments, an enzyme coding sequence encoding the CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

In general, a guide sequence is any polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more.

Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), Clustal W, Clustal X, BLAT, Novoalign (Novocraft Technologies, ELAND (Illumina, San Diego, Calif.), SOAP (available at soap.genomics.org.cn), and Maq (available at maq.sourceforge.net).

The CRISPR enzyme may be part of a fusion protein comprising one or more heterologous protein domains. A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A CRISPR enzyme may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4A DNA binding domain fusions, and herpes simplex virus (HSV) BP16 protein fusions. Additional domains that may form part of a fusion protein comprising a CRISPR enzyme are described in US 20110059502, incorporated herein by reference.

### D. Therapeutic Proteins

Some embodiments concern expression of recombinant proteins and polypeptides, such as those listed below.

Apolipoprotein E2. Apolipoprotein E (APOE) is a protein involved in the metabolism of fats in the body of mammals. A subtype is implicated in Alzheimer's disease and cardiovascular disease. APOE belongs to a family of fat-binding proteins called apolipoproteins. In the circulation, it is present as part of several classes of lipoprotein particles, including chylomicron remnants, VLDL, IDL, and some HDL. APOE interacts significantly with the low-density lipoprotein receptor (LDLR), which is essential for the normal processing (catabolism) of triglyceride-rich lipoproteins. In peripheral tissues, APOE is primarily produced by the liver and macrophages, and mediates cholesterol metabolism. In the central nervous system, APOE is mainly produced by astrocytes and transports cholesterol to neurons via APOE receptors, which are members of the low-density lipoprotein receptor gene family. APOE is the principal cholesterol carrier in the brain. APOE is required for cholesterol transportation from astrocytes to neurons. APOE qualifies as a checkpoint inhibitor of the classical complement pathway by complex formation with activated C1q. APOE is a protein involved in the metabolism of fats in the body of mammals. A subtype is implicated in Alzheimer's disease and cardiovascular disease.

APOE is 299 amino acids long and contains multiple amphipathic α-helices. According to crystallography studies, a hinge region connects the N- and C-terminal regions of the protein. The N-terminal region (residues 1-167) forms an anti-parallel four-helix bundle such that the non-polar sides face inside the protein. Meanwhile, the C-terminal domain (residues 206-299) contains three α-helices which form a large exposed hydrophobic surface and interact with those in the N-terminal helix bundle domain through hydrogen bonds and salt-bridges. The C-terminal region also contains a low-density lipoprotein receptor (LDLR)-binding site.

APOE is polymorphic, with three major alleles (epsilon 2, epsilon 3, and epsilon 4): APOE-ε2 (cys112, cys158), APOE-ε3 (cys112, argl58), and APOE-ε4 (arg112, arg158). Although these allelic forms differ from each other by only one or two amino acids at positions 112 and 158, these differences alter APOE structure and function.

As of 2012, the E4 variant was the largest known genetic risk factor for late-onset sporadic Alzheimer's disease (AD) in a variety of ethnic groups. However, the E4 variant does not correlate with risk in every population. Nigerian people have the highest observed frequency of the APOE4 allele in world populations, but AD is rare among them. This may be due to their low cholesterol levels. Caucasian and Japanese carriers of two E4 alleles have between 10 and 30 times the risk of developing AD by 75 years of age, as compared to those not carrying any E4 alleles. This may be caused by an interaction with amyloid. Alzheimer's disease is characterized by build-ups of aggregates of the peptide beta-amyloid. Apolipoprotein E enhances proteolytic break-down of this peptide, both within and between cells. The isoform APOE-ε4 is not as effective as the others at promoting these reactions, resulting in increased vulnerability to AD in individuals with that gene variation.

Although 40-65% of AD patients have at least one copy of the ε4 allele, APOE4 is not a determinant of the disease. At least one-third of patients with AD are APOE4 negative and some APOE4 homozygotes never develop the disease. Yet those with two ε4 alleles have up to 20 times the risk of developing AD. There is also evidence that the APOE2 allele may serve a protective role in AD. Thus, the genotype most at risk for Alzheimer's disease and at an earlier age is APOE4,4. Using genotype APOE3,3 as a benchmark (with the persons who have this genotype regarded as having a risk level of 1.0) and for white populations only, individuals with genotype APOE4,4 have an odds ratio of 14.9 of developing Alzheimer's disease. Individuals with the APOE3,4 genotype face an odds ratio of 3.2, and people with a copy of the 2 allele and the 4 allele (APOE2,4), have an odds ratio of 2.6. Persons with one copy each of the 2 allele and the 3 allele (APOE2,3) have an odds ratio of 0.6. Persons with two copies of the 2 allele (APOE2,2) also have an odds ratio of 0.6.

Further exemplary therapeutic proteins include secreted antibodies, nanobodies, tripeptidyl peptidase 1 (TPP1), sulfamidase (SGSH), palmitoyl-protein thioesterase 1 (PPT1), beta-glucuronidase (GUSB), alpha-L-iduronidase (IDUA), galactocerebrosidase (GALC), CLN6 transmembrane ER protein (CLN6), beta-galactosidase (GLB1), beta-hexosaminidase A alpha subunit (HEXA), sulfatase modifying factor 1 (SUMF1), alpha-d-mannosidase (MAN2B1), N-acetylglucosamine-6-sulfatase (GNS), heparan-alpha-glucosaminide N-acetyltransferase (HGSNAT), and alpha-N-acetylglucosaminidase (NAGLU).

When the present application refers to the function or activity of “modified protein” or a “modified polypeptide,” one of ordinary skill in the art would understand that this includes, for example, a protein or polypeptide that possesses an additional advantage over the unmodified protein or polypeptide. It is specifically contemplated that embodiments concerning a “modified protein” may be implemented with respect to a “modified polypeptide,” and vice versa.

Recombinant proteins may possess deletions and/or substitutions of amino acids; thus, a protein with a deletion, a protein with a substitution, and a protein with a deletion and a substitution are modified proteins. In some embodiments, these proteins may further include insertions or added amino acids, such as with fusion proteins or proteins with linkers, for example. A “modified deleted protein” lacks one or more residues of the native protein but may possess the specificity and/or activity of the native protein. A “modified deleted protein” may also have reduced immunogenicity or antigenicity. An example of a modified deleted protein is one that has an amino acid residue deleted from at least one antigenic region, i.e. a region of the protein determined to be antigenic in aparticular organism, such as the organism to which the modified protein is being administered.

Substitution or replacement variants typically contain the exchange of one amino acid for another at one or more sites within the protein and may be designed to modulate one or more properties of the polypeptide, particularly its effector functions and/or bioavailability. Substitutions may or may not be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine, or methionine; serine to threonine; threonine to serine; tryptophan to tyrosine; tyrosine to tryptophan orphenylalanine; and valine to isoleucine or leucine.

In addition to a deletion or substitution, a modified protein may possess an insertion of residues, which typically involves the addition of at least one residue in the polypeptide. This may include the insertion of a targeting peptide or polypeptide or simply a single residue. Terminal additions, called fusion proteins, are discussed below.

Gonzalez et al., BMC Neurosci 10.1186/1471-2202-12-4 (2011) screened a peptide library on M13 bacteriophage for ligands capable of internalizing into CP epithelial cells by incubating phage with choroid plexus explants and recovering particles with targeting capacity. Three peptides, identified after four rounds of screening, were analyzed for specific and dose dependent binding and internalization. Binding was deemed specific because internalization was prevented by co-incubation with cognate synthetic peptides. Furthermore, after i.e.v. injection into rat brains, each peptide was found to target phage to epithelial cells in CP and to ependyma lining the ventricles. Such peptides can be employed in the AAV vectors described herein.

The term “biologically functional equivalent” is well understood in the art and is further defined in detail herein. Accordingly, sequences that have between about 70% and about 80%, or between about 81% and about 90%, or even between about 91% and about 99% of amino acids that are identical or functionally equivalent to the amino acids of a control polypeptide are included, provided the biological activity of the protein is maintained. A recombinant protein may be biologically functionally equivalent to its native counterpart in certain aspects.

It also will be understood that amino acid and nucleic acid sequences may include additional residues, such as additional N- or C-terminal amino acids or 5′ or 3′ sequences, and yet still be essentially as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region or may include various internal sequences, i.e., introns, which are known to occur within genes.

As used herein, a protein or peptide generally refers, but is not limited to, a protein of greater than about 200 amino acids, up to a full-length sequence translated from a gene; a polypeptide of greater than about 100 amino acids; and/or a peptide of from about 3 to about 100 amino acids. For convenience, the terms “protein,” “polypeptide,” and “peptide are used interchangeably herein.

As used herein, an “amino acid residue” refers to any naturally occurring amino acid, any amino acid derivative, or any amino acid mimic known in the art. In certain embodiments, the residues of the protein or peptide are sequential, without any non-amino acids interrupting the sequence of amino acid residues. In other embodiments, the sequence may comprise one or more non-amino acid moieties. In particular embodiments, the sequence of residues of the protein or peptide may be interrupted by one or more non-amino acid moieties.

Accordingly, the term “protein or peptide” encompasses amino acid sequences comprising at least one of the 20 common amino acids found in naturally occurring proteins, or at least one modified or unusual amino acid.

Certain embodiments of the present disclosure concern fusion proteins. These molecules may have a therapeutic protein linked at the N- or C-terminus to a heterologous domain. For example, fusions may also employ leader sequences from other species to permit the recombinant expression of a protein in a heterologous host. Another useful fusion includes the addition of a protein affinity tag, such as a serum albumin affinity tag or six histidine residues, or an immunologically active domain, such as an antibody epitope, preferably cleavable, to facilitate purification of the fusion protein. Non-limiting affinity tags include polyhistidine, chitin binding protein (CBP), maltose binding protein (MBP), and glutathione-S-transferase (GST).

Methods of generating fusion proteins are well known to those of skill in the art. Such proteins can be produced, for example, by de novo synthesis of the complete fusion protein, or by attachment of the DNA sequence encoding the heterologous domain, followed by expression of the intact fusion protein.

Production of fusion proteins that recover the functional activities of the parent proteins may be facilitated by connecting genes with a bridging DNA segment encoding a peptide linker that is spliced between the polypeptides connected in tandem. The linker would be of sufficient length to allow proper folding of the resulting fusion protein.

### III. METHODS OF TREATMENT AND ADMINISTRATION

Viral vectors may, in some aspects, be administered directly to patients (in vivo) or they can be used to treat cells in vitro or ex vivo, and then administered to patients. In particular, provided herein are methods for inducing expression of a transgene in the ependyma. In some of these embodiments, the subject has a brain or neurological disorder, and the transgene is delivered in a therapeutically effective amount. In some embodiments, the AAV vector transduces at least about 70% of cells of the target tissue; the AVV targets inner and outer hair cells with at least about 70%, 80%, 90%, 95% or greater efficiency, even as high as 100% efficiency. In some embodiments, the cell is a cell of the ventricles of the brain, e.g., an ependymal cell.

The ependyma is the thin neuroepithelial (simple columnar ciliated epithelium) lining of the ventricular system of the brain and the central canal of the spinal cord. The ependyma is one of the four types of neuroglia in the central nervous system (CNS). It is involved in the production of cerebrospinal fluid (CSF) and is shown to serve as a reservoir for neuroregeneration. The ependyma is made up of ependymal cells called ependymocytes, a type of glial cell. These cells line the ventricles in the brain and the central canal of the spinal cord, which become filled with cerebrospinal fluid. These are nervous tissue cells with simple columnar shape, much like that of some mucosal epithelial cells. Early monociliated ependymal cells are differentiated to multiciliated ependymal cells for their function in circulating cerebrospinal fluid. The basal membranes of these cells are characterized by tentacle-like extensions that attach to astrocytes. The apical side is covered in cilia and microvilli.

The term “vector” refers to small carrier nucleic acid molecule, a plasmid, virus (e.g., AAV vector, retroviral vector, lentiviral vector), or other vehicle that can be manipulated by insertion or incorporation of a nucleic acid. Vectors, such as viral vectors, can be used to introduce/transfer nucleic acid sequences into cells, such that the nucleic acid sequence therein is transcribed and, if encoding a protein, subsequently translated by the cells.

These compositions can be used to treat a condition of the brain or central nervous system. Thus, in some embodiments, the methods described herein are used to treat a condition listed in Table A, using the corresponding sequence listing in Table A, in a subject in need thereof.

Any suitable cell or mammal can be administered or treated by a method or use described herein. Typically, a mammal in need of a method described herein is suspected of having or expressing an abnormal or aberrant protein that is associated with a disease state. Alternatively, the mammalian recipient may have a condition that is amenable to gene replacement therapy. As used herein, “gene replacement therapy” refers to administration to the recipient of exogenous genetic material encoding a therapeutic agent and subsequent expression of the administered genetic material in situ. Thus, the phrase “condition amenable to gene replacement therapy” embraces conditions such as genetic diseases (i.e., a disease condition that is attributable to one or more gene defects) and acquired pathologies (i.e., a pathological condition which is not attributable to an inborn defect). Accordingly, as used herein, the term “therapeutic agent” refers to any agent or material which has a beneficial effect on the mammalian recipient. Thus, “therapeutic agent” embraces both therapeutic and prophylactic molecules having nucleic acid or protein components.

Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In certain embodiments a mammal is a human. In certain embodiments a mammal is a non-rodent mammal (e.g., human, pig, goat, sheep, horse, dog, or the like). In certain embodiments a non-rodent mammal is a human. A mammal can be any age or at any stage of development (e.g., an adult, teen, child, infant, or a mammal in utero). A mammal can be male or female. In certain embodiments a mammal can be an animal disease model, for example, animal models having or expressing an abnormal or aberrant protein that is associated with a disease state or animal models with insufficient expression of a protein, which causes a disease state.

Mammals (subjects) treated by a method or composition described herein include adults (18 years or older) and children (less than 18 years of age). Adults include the elderly. Representative adults are 50 years or older. Children range in age from 1-2 years old, or from 2-4, 4-6, 6-18, 8-10, 10-12, 12-15 and 15-18 years old. Children also include infants. Infants typically range from 1-12 months of age.

In certain embodiments, a method includes administering a plurality of viral particles to a mammal as set forth herein, where severity, frequency, progression or time of onset of one or more symptoms of a disease state, such as a neuro-degenerative disease, decreased, reduced, prevented, inhibited or delayed. In certain embodiments, a method includes administering a plurality of viral particles to a mammal to treat an adverse symptom of a disease state, such as a neuro-degenerative disease. In certain embodiments, a method includes administering a plurality of viral particles to a mammal to stabilize, delay or prevent worsening, or progression, or reverse and adverse symptom of a disease state, such as a neuro-degenerative disease.

In certain embodiments a method includes administering a plurality of viral particles to the central nervous system, or portion thereof as set forth herein, of a mammal and severity, frequency, progression or time of onset of one or more symptoms of a disease state, such as a neuro-degenerative disease, are decreased, reduced, prevented, inhibited or delayed by at least about 5 to about 10, about 10 to about 25, about 25 to about 50, or about 50 to about 100 days.

In some embodiments, a composition comprising a therapeutically effective number of virus particles containing a transgene, or containing one or more sets of different virus particles, wherein each particle in a set can contain the same type of transgene, but wherein each set of particles contains a different type of transgene than in the other sets, as described herein can be delivered.

Formulations according to the present disclosure can be used for CNS delivery via various techniques and routes including, but not limited to, intraparenchymal, intracerebral, intravetricular cerebral (ICV), intrathecal (e.g., IT-Lumbar, IT-thoracic, IT-cisterna magna) administrations and any other techniques and routes for injection directly or indirectly to the CNS and/or CSF.

In some embodiments, a formulation is delivered to the CNS by administering into the cerebrospinal fluid (CSF) of a subject in need of treatment. In some embodiments, intrathecal administration is used to deliver viral particles into the CSF. As used herein, intrathecal administration (also referred to as intrathecal injection) refers to an injection into the spinal canal (intrathecal space surrounding the spinal cord). Various techniques may be used including, without limitation, lateral cerebroventricular injection through a burrhole or cisternal or lumbar puncture or the like. Exemplary methods are described in Lazorthes et al. Advances in Drug Delivery Systems and Applications in Neurosurgery, 18:143-192 (1991) and Ommaya et al., Cancer Drug Delivery, 1:169-179 (1984) the contents of which are incorporated herein by reference.

According to the present disclosure, viral particles may be injected at any region surrounding the spinal canal. In some embodiments, viral particles are injected into the lumbar area or the cisterna magna or intraventricularly into a cerebral ventricle space. As used herein, the term “lumbar region” or “lumbar area” refers to the area between the third and fourth lumbar (lower back) vertebrae and, more inclusively, the L2-S 1 region of the spine. Typically, intrathecal injection via the lumbar region or lumber area is also referred to as “lumbar IT delivery” or “lumbar IT administration.” The term “cisterna magna” refers to the space around and below the cerebellum via the opening between the skull and the top of the spine. Typically, intrathecal injection via cisterna magna is also referred to as “cisterna magna delivery.” The term “cerebral ventricle” refers to the cavities in the brain that are continuous with the central canal of the spinal cord. As such, intrathecal administration includes any infusion into the central canal. Typically, injections via the cerebral ventricle cavities are referred to as intravetricular cerebral (ICV) delivery.

Various devices may be used for intrathecal delivery according to the present disclosure. In some embodiments, a device for intrathecal administration contains a fluid access port (e.g., injectable port); a hollow body (e.g., catheter) having a first flow orifice in fluid communication with the fluid access port and a second flow orifice configured for insertion into spinal cord; and a securing mechanism for securing the insertion of the hollow body in the spinal cord. In various embodiments, the fluid access port comprises a reservoir. In some embodiments, the fluid access port comprises a mechanical pump (e.g., an infusion pump). In some embodiments, an implanted catheter is connected to either a reservoir (e.g., for bolus delivery), or an infusion pump. The fluid access port may be implanted or external.

In some embodiments, intrathecal administration may be performed by either lumbar puncture (i.e., slow bolus) or via a port-catheter delivery system (i.e., infusion or bolus). In some embodiments, the catheter is inserted between the laminae of the lumbar vertebrae and the tip is threaded up the thecal space to the desired level (generally L3-L4).

A single dose volume suitable for intrathecal administration is typically small. Typically, intrathecal delivery according to the present disclosure maintains the balance of the composition of the CSF as well as the intracranial pressure of the subject. In some embodiments, intrathecal delivery is performed absent the corresponding removal of CSF from a subject. In some embodiments, a suitable single dose volume may be e.g., less than about 10 ml, 8 ml, 6 ml, 5 ml, 4 ml, 3 ml, 2 ml, 1.5 ml, 1 ml, or 0.5 ml. In some embodiments, a suitable single dose volume may be about 0.5-5 ml, 0.5-4 ml, 0.5-3 ml, 0.5-2 ml, 0.5-1 ml, 1-3 ml, 1-5 ml, 1.5-3 ml, 1-4 ml, or 0.5-1.5 ml. In some embodiments, intrathecal delivery according to the present disclosure involves a step of removing a desired amount of CSF first. In some embodiments, less than about 10 ml (e.g., less than about 9 ml, 8 ml, 7 ml, 6 ml, 5 ml, 4 ml, 3 ml, 2 ml, 1 ml) of CSF is first removed before IT administration. In those cases, a suitable single dose volume may be e.g., more than about 3 ml, 4 ml, 5 ml, 6 ml, 7 ml, 8 ml, 9 ml, 10 ml, 15 ml, or 20 ml.

Various other devices may be used to effect intrathecal administration of a therapeutic composition. For example, formulations containing desired enzymes may be given using an Ommaya reservoir which is in common use for intrathecally administering drugs for meningeal carcinomatosis (Ommaya, Lancet 2: 983-84, 1963). More specifically, in this method, a ventricular tube is inserted through a hole formed in the anterior horn and is connected to an Ommaya reservoir installed under the scalp, and the reservoir is subcutaneously punctured to intrathecally deliver the particular enzyme being replaced, which is injected into the reservoir. Other devices for intrathecal administration of therapeutic compositions or formulations to an individual are described in U.S. Pat. No. 6,217,552, incorporated herein by reference. Alternatively, the viral particles may be intrathecally given, for example, by a single injection, or continuous infusion. It should be understood that the dosage treatment may be in the form of a single dose administration or multiple doses.

In one embodiment of the disclosure, the viral particles are administered by lateral cerebro ventricular injection into the brain of a subject. The injection can be made, for example, through a burr hole made in the subject's skull. In another embodiment, the viral particles and/or other pharmaceutical formulation are administered through a surgically inserted shunt into the cerebral ventricle of a subject. For example, the injection can be made into the lateral ventricles, which are larger. In some embodiments, injection into the third and fourth smaller ventricles can also be made. In yet another embodiment, the pharmaceutical compositions used in the present disclosure are administered by injection into the cisterna magna, or lumbar area of a subject.

### IV. PHARMACEUTICAL COMPOSITIONS

As used herein the term “pharmaceutically acceptable” and “physiologically acceptable” mean a biologically acceptable composition, formulation, liquid or solid, or mixture thereof, which is suitable for one or more routes of administration, in vivo delivery or contact. A “pharmaceutically acceptable” or “physiologically acceptable” composition is a material that is not biologically or otherwise undesirable, e.g., the material may be administered to a subject without causing substantial undesirable biological effects. Such composition, “pharmaceutically acceptable” and “physiologically acceptable” formulations and compositions can be sterile. Such pharmaceutical formulations and compositions may be used, for example in administering a viral particle to a subject.

Such formulations and compositions include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Supplementary active compounds (e.g., preservatives, antibacterial, antiviral and antifungal agents) can also be incorporated into the formulations and compositions.

Pharmaceutical compositions typically contain a pharmaceutically acceptable excipient. Such excipients include any pharmaceutical agent that does not itself induce the production of antibodies harmful to the individual receiving the composition, and which may be administered without undue toxicity. Pharmaceutically acceptable excipients include, but are not limited to, sorbitol, Tween80, and liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as surfactants, wetting or emulsifying agents, pH buffering substances, and the like, may be present in such vehicles.

Pharmaceutical compositions can be formulated to be compatible with a particular route of administration or delivery, as set forth herein or known to one of skill in the art. Thus, pharmaceutical compositions include carriers, diluents, or excipients suitable for administration or delivery by various routes.

Pharmaceutical forms suitable for injection or infusion of viral particles can include sterile aqueous solutions or dispersions which are adapted for the extemporaneous preparation of sterile injectable or infusible solutions or dispersions, optionally encapsulated in liposomes. In all cases, the ultimate form should be a sterile fluid and stable under the conditions of manufacture, use and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Isotonic agents, for example, sugars, buffers or salts (e.g., sodium chloride) can be included. Prolonged absorption of injectable compositions can be brought about by the use in the compositions of agents delaying absorption, for example, aluminum monostearate and gelatin.

Solutions or suspensions of viral particles can optionally include one or more of the following components: a sterile diluent such as water for injection, saline solution, such as phosphate buffered saline (PBS), artificial CSF, a surfactants, fixed oils, a polyol (for example, glycerol, propylene glycol, and liquid polyethylene glycol, and the like), glycerin, or other synthetic solvents; antibacterial and antifungal agents such as parabens, chlorobutanol, phenol, ascorbic acid, and the like; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose.

Pharmaceutical formulations, compositions and delivery systems appropriate for the compositions, methods and uses of the disclosure are known in the art (see, e.g., Remington: The Science and Practice of Pharmacy (2003) 20^{th} ed., Mack Publishing Co., Easton, PA; Remington's Pharmaceutical Sciences (1990) 18^{th} ed., Mack Publishing Co., Easton, PA; The Merck Index (1996) 12^{th} ed., Merck Publishing Group, Whitehouse, NJ; Pharmaceutical Principles of Solid Dosage Forms (1993), Technonic Publishing Co., Inc., Lancaster, Pa.; Ansel and Stoklosa, Pharmaceutical Calculations (2001) 11^{th} ed., Lippincott Williams & Wilkins, Baltimore, MD; and Poznansky et al., Drug Delivery Systems (1980), R. L. Juliano, ed., Oxford, N.Y., pp. 253-315).

Viral particles and their compositions may be formulated in dosage unit form for ease of administration and uniformity of dosage. Dosage unit form as used herein refers to physically discrete units suited as unitary dosages for an individual to be treated; each unit containing a predetermined quantity of active compound calculated to produce the desired therapeutic effect in association with the required pharmaceutical carrier. The dosage unit forms are dependent upon the number of viral particles believed necessary to produce the desired effect(s). The amount necessary can be formulated in a single dose or can be formulated in multiple dosage units. The dose may be adjusted to a suitable viral particle concentration, optionally combined with an anti-inflammatory agent, and packaged for use.

In one embodiment, pharmaceutical compositions will include sufficient genetic material to provide a therapeutically effective amount, i.e., an amount sufficient to reduce or ameliorate symptoms or an adverse effect of a disease state in question or an amount sufficient to confer the desired benefit.

A “unit dosage form” as used herein refers to physically discrete units suited as unitary dosages for the subject to be treated; each unit containing a predetermined quantity optionally in association with a pharmaceutical carrier (excipient, diluent, vehicle or filling agent) which, when administered in one or more doses, is calculated to produce a desired effect (e.g., prophylactic or therapeutic effect). Unit dosage forms may be within, for example, ampules and vials, which may include a liquid composition, or a composition in a freeze-dried or lyophilized state; a sterile liquid carrier, for example, can be added prior to administration or delivery in vivo. Individual unit dosage forms can be included in multi-dose kits or containers. Thus, for example, viral particles, and pharmaceutical compositions thereof, can be packaged in single or multiple unit dosage form for ease of administration and uniformity of dosage.

Formulations containing viral particles typically contain an effective amount, the effective amount being readily determined by one skilled in the art. The viral particles may typically range from about 1% to about 95% (w/w) of the composition, or even higher if suitable. The quantity to be administered depends upon factors such as the age, weight and physical condition of the mammal or the human subject considered for treatment. Effective dosages can be established by one of ordinary skill in the art through routine trials establishing dose response curves.

### V. DEFINITIONS

The terms “polynucleotide,” “nucleic acid” and “transgene” are used interchangeably herein to refer to all forms of nucleic acid, oligonucleotides, including deoxyribonucleic acid (DNA) and ribonucleic acid (RNA) and polymers thereof. Polynucleotides include genomic DNA, cDNA and antisense DNA, and spliced or unspliced mRNA, rRNA, tRNA and inhibitory DNA or RNA (RNAi, e.g., small or short hairpin (sh)RNA, microRNA (miRNA), small or short interfering (si)RNA, trans-splicing RNA, or antisense RNA). Polynucleotides can include naturally occurring, synthetic, and intentionally modified or altered polynucleotides (e.g., variant nucleic acid). Polynucleotides can be single stranded, double stranded, or triplex, linear or circular, and can be of any suitable length. In discussing polynucleotides, a sequence or structure of a particular polynucleotide may be described herein according to the convention of providing the sequence in the 5′ to 3′ direction.

A nucleic acid encoding a polypeptide often comprises an open reading frame that encodes the polypeptide. Unless otherwise indicated, a particular nucleic acid sequence also includes degenerate codon substitutions.

Nucleic acids can include one or more expression control or regulatory elements operably linked to the open reading frame, where the one or more regulatory elements are configured to direct the transcription and translation of the polypeptide encoded by the open reading frame in a mammalian cell. Non-limiting examples of expression control/regulatory elements include transcription initiation sequences (e.g., promoters, enhancers, a TATA box, and the like), translation initiation sequences, mRNA stability sequences, poly A sequences, secretory sequences, and the like. Expression control/regulatory elements can be obtained from the genome of any suitable organism.

A “promoter” refers to a nucleotide sequence, usually upstream (5′) of a coding sequence, which directs and/or controls the expression of the coding sequence by providing the recognition for RNA polymerase and other factors required for proper transcription. A pol II promoter includes a minimal promoter that is a short DNA sequence comprised of a TATA-box and optionally other sequences that serve to specify the site of transcription initiation, to which regulatory elements are added for control of expression. A type 1 pol III promoter includes three cis-acting sequence elements downstream of the transcriptional start site: a) 5′sequence element (A block); b) an intermediate sequence element (I block); c) 3′ sequence element (C block). A type 2 pol III promoter includes two essential cis-acting sequence elements downstream of the transcription start site: a) an A box (5′ sequence element); and b) a B box (3′ sequence element). A type 3 pol III promoter includes several cis-acting promoter elements upstream of the transcription start site, such as a traditional TATA box, proximal sequence element (PSE), and a distal sequence element (DSE).

An “enhancer” is a DNA sequence that can stimulate transcription activity and may be an innate element of the promoter or a heterologous element that enhances the level or tissue specificity of expression. It is capable of operating in either orientation (5′->3′ or 3′->5′), and may be capable of functioning even when positioned either upstream or downstream of the promoter.

Enhancers may be derived in their entirety from a native gene or be composed of different elements derived from different elements found in nature, or even be comprised of synthetic DNA segments. An enhancer may comprise DNA sequences that are involved in the binding of protein factors that modulate/control effectiveness of transcription initiation in response to stimuli, physiological or developmental conditions.

A “transgene” is used herein to conveniently refer to a nucleic acid sequence/polynucleotide that is intended or has been introduced into a cell or organism. Transgenes include any nucleic acid, such as a gene that encodes an inhibitory RNA or polypeptide or protein and are generally heterologous with respect to naturally occurring AAV genomic sequences.

The term “transduce” refers to introduction of a nucleic acid sequence into a cell or host organism by way of a vector (e.g., a viral particle). Introduction of a transgene into a cell by a viral particle can therefore be referred to as “transduction” of the cell. The transgene may or may not be integrated into genomic nucleic acid of a transduced cell. If an introduced transgene becomes integrated into the nucleic acid (genomic DNA) of the recipient cell or organism it can be stably maintained in that cell or organism and further passed on to or inherited by progeny cells or organisms of the recipient cell or organism. Finally, the introduced transgene may exist in the recipient cell or host organism extra chromosomally, or only transiently. A “transduced cell” is therefore a cell into which the transgene has been introduced by way of transduction. Thus, a “transduced” cell is a cell into which, or a progeny thereof in which a transgene has been introduced. A transduced cell can be propagated, a transgene transcribed and the encoded inhibitory RNA or protein expressed. For gene therapy uses and methods, a transduced cell can be in a mammal.

A nucleic acid/transgene is “operably linked” when it is placed into a functional relationship with another nucleic acid sequence, where the promoter is capable of controlling transcription of the encoded polypeptide. A nucleic acid operably linked to an expression control element can also be referred to as an expression cassette.

In certain embodiments, an expression control element comprises a CMV enhancer.

As used herein, the terms “modify” or “variant” and grammatical variations thereof, mean that a nucleic acid, polypeptide or subsequence thereof deviates from a reference sequence. Modified and variant sequences may therefore have substantially the same, greater or less expression, activity or function than a reference sequence, but at least retain partial activity or function of the reference sequence. A particular type of variant is a mutant protein, which refers to a protein encoded by a gene having a mutation, e.g., a missense or nonsense mutation.

A “nucleic acid” or “polynucleotide” variant refers to a modified sequence which has been genetically altered compared to wild-type. The sequence may be genetically modified without altering the encoded protein sequence. Alternatively, the sequence may be genetically modified to encode a variant protein. A nucleic acid or polynucleotide variant can also refer to a combination sequence which has been codon modified to encode a protein that still retains at least partial sequence identity to a reference sequence, such as wild-type protein sequence, and also has been codon-modified to encode a variant protein. For example, some codons of such a nucleic acid variant will be changed without altering the amino acids of a protein encoded thereby, and some codons of the nucleic acid variant will be changed which in turn changes the amino acids of a protein encoded thereby.

The terms “protein” and “polypeptide” are used interchangeably herein. The “polypeptides” encoded by a “nucleic acid” or “polynucleotide” or “transgene” disclosed herein include partial or full-length native sequences, as with naturally occurring wild-type and functional polymorphic proteins, functional subsequences (fragments) thereof, and sequence variants thereof, so long as the polypeptide retains some degree of function or activity. Accordingly, in methods and uses of the disclosure, such polypeptides encoded by nucleic acid sequences are not required to be identical to the endogenous protein that is defective, or whose activity, function, or expression is insufficient, deficient or absent in a treated mammal.

Non-limiting examples of modifications include one or more nucleotide or amino acid substitutions (e.g., about 1 to about 3, about 3 to about 5, about 5 to about 10, about 10 to about 15, about 15 to about 20, about 20 to about 25, about 25 to about 30, about 30 to about 40, about 40 to about 50, about 50 to about 100, about 100 to about 150, about 150 to about 200, about 200 to about 250, about 250 to about 500, about 500 to about 750, about 750 to about 1000 or more nucleotides or residues).

An example of an amino acid modification is a conservative amino acid substitution or a deletion. In particular embodiments, a modified or variant sequence retains at least part of a function or activity of the unmodified sequence (e.g., wild-type sequence).

Another example of an amino acid modification is a targeting peptide introduced into a capsid protein of a viral particle. Peptides have been identified that target recombinant viral vectors, to the central nervous system, such as to distinct brain regions.

A recombinant virus so modified may preferentially bind to one type of tissue (e.g., CNS tissue) over another type of tissue (e.g., liver tissue). In certain embodiments, a recombinant virus bearing a modified capsid protein may “target” brain vascular epithelia tissue by binding at level higher than a comparable, unmodified capsid protein. For example, a recombinant virus having a modified capsid protein may bind to brain ependymal tissue at a level 50% to 100% greater than an unmodified recombinant virus.

A “nucleic acid fragment” is a portion of a given nucleic acid molecule. Deoxyribonucleic acid (DNA) in the majority of organisms is the genetic material while ribonucleic acid (RNA) is involved in the transfer of information contained within DNA into proteins. Fragments and variants of the disclosed nucleotide sequences and proteins or partial-length proteins encoded thereby are also encompassed by the present disclosure. “Fragment” or “portion” means a full length or less than full length of the nucleotide sequence encoding, or the amino acid sequence of, a polypeptide or protein. In certain embodiments, the fragment or portion is biologically functional (i.e., retains 5%, 10%, 15%, 20%, 25%, 30%, 35%, 40%, 45%, 50%, 55%, 60%, 65%, 70%, 75%, 80%, 85%, 90%, 95%, 99% or 100% of activity or function of wild-type).

A “variant” of a molecule is a sequence that is substantially similar to the sequence of the native molecule. For nucleotide sequences, variants include those sequences that, because of the degeneracy of the genetic code, encode the identical amino acid sequence of the native protein. Naturally occurring allelic variants such as these can be identified with the use of molecular biology techniques, as, for example, with polymerase chain reaction (PCR) and hybridization techniques. Variant nucleotide sequences also include synthetically derived nucleotide sequences, such as those generated, for example, by using site-directed mutagenesis, which encode the native protein, as well as those that encode a polypeptide having amino acid substitutions. Generally, nucleotide sequence variants of the disclosure will have at least 40%, 50%, 60%, to 70%, e.g., 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98%, sequence identity to the native (endogenous) nucleotide sequence. In certain embodiments, the variant is biologically functional (i.e., retains 5%, 10%, 15%, 20%, 25%, 30%, 35%, 40%, 45%, 50%, 55%, 60%, 65%, 70%, 75%, 80%, 85%, 90%, 95%, 99% or 100% of activity or function of wild-type).

“Conservative variations” of a particular nucleic acid sequence refers to those nucleic acid sequences that encode identical or essentially identical amino acid sequences. Because of the degeneracy of the genetic code, a large number of functionally identical nucleic acids encode any given polypeptide. For instance, the codons CGT, CGC, CGA, CGG, AGA and AGG all encode the amino acid arginine. Thus, at every position where an arginine is specified by a codon, the codon can be altered to any of the corresponding codons described without altering the encoded protein. Such nucleic acid variations are “silent variations,” which are one species of “conservatively modified variations.” Every nucleic acid sequence described herein that encodes a polypeptide also describes every possible silent variation, except where otherwise noted. One of skill in the art will recognize that each codon in a nucleic acid (except ATG, which is ordinarily the only codon for methionine) can be modified to yield a functionally identical molecule by standard techniques. Accordingly, each “silent variation” of a nucleic acid that encodes a polypeptide is implicit in each described sequence.

The term “substantial identity” of polynucleotide sequences means that a polynucleotide comprises a sequence that has at least 70%, 71%, 72%, 73%, 74%, 75%, 76%, 77%, 78%, or 79%, or at least 80%, 81%, 82%, 83%, 84%, 85%, 86%, 87%, 88%, or 89%, or at least 90%, 91%, 92%, 93%, or 94%, or even at least 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 70%, at least 80%, 90%, or even at least 95%.

The term “substantial identity” in the context of a polypeptide indicates that a polypeptide comprises a sequence with at least 70%, 71%, 72%, 73%, 74%, 75%, 76%, 77%, 78%, or 79%, or 80%, 81%, 82%, 83%, 84%, 85%, 86%, 87%, 88%, or 89%, or at least 90%, 91%, 92%, 93%, or 94%, or even, 95%, 96%, 97%, 98% or 99%, sequence identity to the reference sequence over a specified comparison window. An indication that two polypeptide sequences are identical is that one polypeptide is immunologically reactive with antibodies raised against the second polypeptide. Thus, a polypeptide is identical to a second polypeptide, for example, where the two peptides differ only by a conservative substitution.

“Disease” means any condition or disorder that damages or interferes with the normal function of a cell, tissue, or organ.

The terms “treat” and “treatment” refer to both therapeutic treatment and prophylactic or preventative measures, wherein the object is to prevent, inhibit, reduce, or decrease an undesired physiological change or disorder, such as the development, progression or worsening of the disorder. For purposes of this disclosure, beneficial or desired clinical results include, but are not limited to, alleviation of symptoms, diminishment of extent of disease, stabilizing a (i.e., not worsening or progressing) symptom or adverse effect of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those predisposed (e.g., as determined by a genetic assay).

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the inherent variation in the method being employed to determine the value, the variation that exists among the study subjects, or a value that is within 10% of a stated value.

### VI. KITS

The disclosure provides kits with packaging material and one or more components therein. A kit typically includes a label or packaging insert including a description of the components or instructions for use in vitro, in vivo, or ex vivo, of the components therein. A kit can contain a collection of such components, e.g., a nucleic acid, recombinant vector, and/or viral particles.

A kit refers to a physical structure housing one or more components of the kit. Packaging material can maintain the components sterilely,and can be made of material commonly used for such purposes (e.g., paper, corrugated fiber, glass, plastic, foil, ampules, vials, tubes, etc.).

Labels or inserts can include identifying information of one or more components therein, dose amounts, clinical pharmacology of the active ingredient(s) including mechanism of action, pharmacokinetics and pharmacodynamics. Labels or inserts can include information identifying manufacturer, lot numbers, manufacture location and date, expiration dates. Labels or inserts can include information identifying manufacturer information, lot numbers, manufacturer location and date. Labels or inserts can include information on a disease for which a kit component may be used. Labels or inserts can include instructions for the clinician or subject for using one or more of the kit components in a method, use, or treatment protocol or therapeutic regimen. Instructions can include dosage amounts, frequency or duration, and instructions for practicing any of the methods, uses, treatment protocols or prophylactic or therapeutic regimes described herein.

Labels or inserts can include information on any benefit that a component may provide, such as a prophylactic or therapeutic benefit. Labels or inserts can include information on potential adverse side effects, complications or reactions, such as warnings to the subject or clinician regarding situations where it would not be appropriate to use a particular composition. Adverse side effects or complications could also occur when the subject has, will be or is currently taking one or more other medications that may be incompatible with the composition, or the subject has, will be or is currently undergoing another treatment protocol or therapeutic regimen which would be incompatible with the composition and, therefore, instructions could include information regarding such incompatibilities.

Labels or inserts include “printed matter,” e.g., paper or cardboard, or separate or affixed to a component, a kit or packing material (e.g., a box), or attached to an ampule, tube or vial containing a kit component. Labels or inserts can additionally include a computer readable medium, such as a bar-coded printed label, a disk, optical disk such as CD- or DVD-ROM/RAM, DVD, MP3, or an electrical storage media such as RAM and ROM or hybrids of these such as magnetic/optical storage media, FLASH memory, hybrids and memory type cards.

### VII. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1

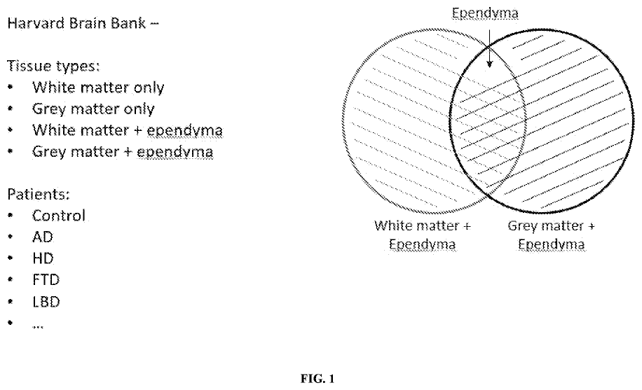

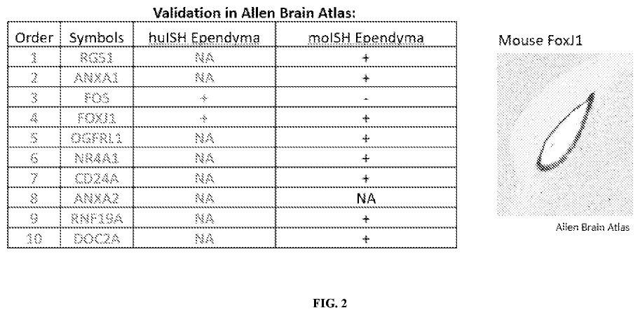

Subtractive approach to identify ependyma-enriched or ependyma-specific genes. The ependyma constitutes a thin epithelial layer that lines the ventricles of the brain and the central canal of the spinal column. These cells are in close proximity to the cerebrospinal fluid (CSF) of the ventricles, a fluid that not only fills these cavities but is distributed widely throughout the brain by trafficking to the subarachnoid space and diffusing along perivascular spaces into the parenchyma. Secreted proteins from the ependyma into the CSF therefore can be delivered broadly throughout the brain. Therapeutically, infection of ependymal cells with adeno-associated viruses (AAV) encoding secreted proteins has been demonstrated to promote widespread protein expression in rodent, dog, and non-human primate brain. To ensure robust, long-term expression of ependyma-localized AAV, the inventors sought to identify endogenous gene signatures and their respective regulatory regions that could be incorporated into AAV transgenes. Important in this search was to identify genes whose expression was insensitive to disease state, ensuring strong expression even when introduced into a pathological brain. As such, the inventors sourced samples from healthy patients as well as ones with Frontotemporal Dementia, semantic dementia, Lewy body dementia, Alzheimer's disease (AD), and Huntington's disease (HD), among others. Due to the limited depth of the ependyma lining, clean isolation of this specific tissue type is very difficult. To circumvent this issue, the inventors employed a subtractive approach to identify ependyma-enriched or ependyma-specific genes. Tissue samples were obtained from ventricle-adjacent white matter and grey matter as well as white matter and grey matter at the ventricle margins containing ependyma. Genes specific to the white matter+ependyma and grey matter+ependyma samples were considered ependyma-enriched or -specific (FIG. 1). Top hits were compared against in situ hybridization data published by the Allen Brain Institute (FIG. 2).

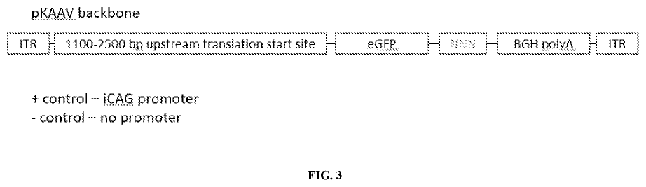

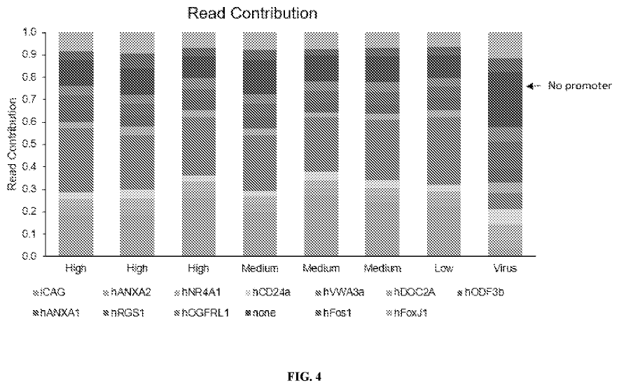

Isolation of predicted regulatory regions in ependyma-specific genes. To isolate functional promoter segments, the UCSC Genome browser was used to search for promoter-like signatures in the upstream sequence of identified genes. Specifically of interest were segments containing H3K4me3, H3K27Ac, CpG islands, and transcription factor binding sites. Approximately 1100-2500 base pair (bp) segments were PCR amplified from human (HT1080) genomic DNA (gDNA) and placed upstream an eGFP reporter. Promoters were identified for high-throughput sequencing using a 3-bp barcode in the 3′ UTR (FIG. 3). Individual plasmids were pooled at equal molar ratios and transgenes were packaged as libraries in AAV4 or AAV2 capsids for testing in mouse and rhesus macaque, respectively.

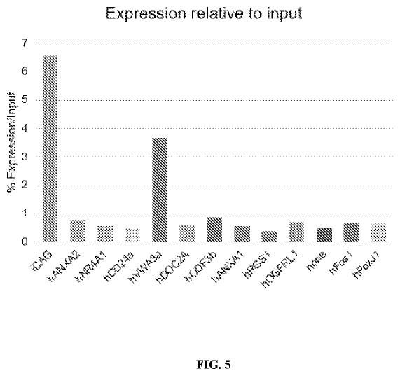

In vivo testing of isolated promoter segments to drive RNA expression in mouse and rhesus ependyma. A library of 13 transgenes containing ependyma-enriched promoters, prepared as a single AAV4, was injected into the lateral ventricle of adult FVBn mice at high (1e11 vg), medium (5e10 vg), and low (1e10 vg) doses. After three weeks, the ventricle lining and limited surrounding tissue were micro-dissected and RNA was isolated using Trizol. Regions surrounding the 3-bp barcodes were amplified from cDNA using a two-step PCR protocol to ligate Illumina sequencing adapters. Final products were subjected to amplicon sequencing to quantify relative contributions. The graph in FIG. 4 demonstrates the fractional contribution of each barcode to the total read counts in the tissue and input virus.

To quantify function of isolated segments as promoters in vivo, the relative contribution of RNA output was normalized to that of the input virus. As expected, the ubiquitous iCAG promoter showed strong expression in vivo (FIG. 5). The transgene containing regulatory sequence from the human Von Willebrand Factor A Domain containing 3a (hVWA3a) gene was also enriched over the viral input.

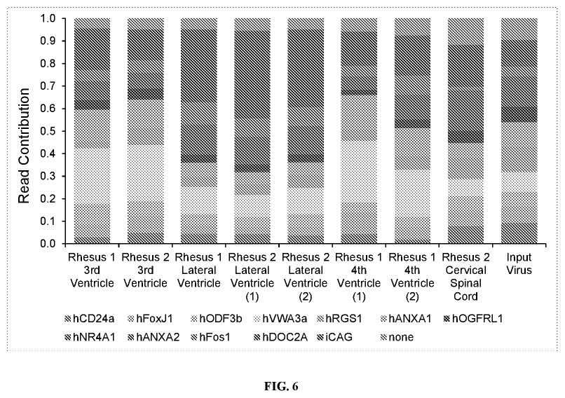

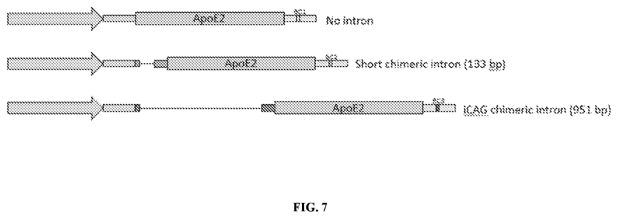

The same library used in FIG. 4 was prepared as AAV2 and injected into the lateral ventricle of two adult rhesus macaques at 2e13 total vg per animal. After three weeks, regions at the ventricle margins, including the spinal cord, were micro-dissected (FIG. 6). RNA was isolated, converted to cDNA and PCR products containing the 3-bp barcode were subjected to amplicon sequencing. Colored bars indicate the relative contribution of each transgene in tissue RNA or viral DNA. Patterns for RNA enrichment in matched locations were similar among the two different macaques and within separate regions of the same structure.

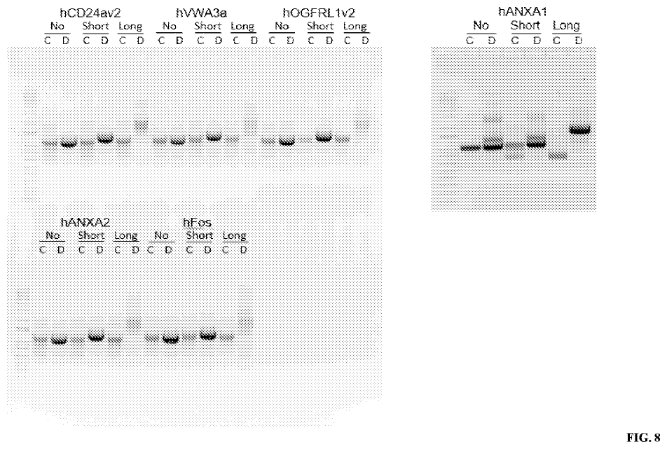

Modification of original transgenes to contain promoter-localized introns and ApoE2 coding sequence. Genetic variation within the apolipoprotein E gene (APOE) on chromosome 19 has been linked to differential risk for late-onset Alzheimer's disease. APOE3, the most common allele is believed to be neutral, neither increasing nor decreasing a person's risk of developing disease. APOE4 increases risk and is linked to earlier disease onset while APOE2, a relatively rare variant, provides protection against disease. Intracerebroventricular injection of AAV4-APOE2 into a mouse model of early-onset AD led to transduction of the ependyma and choroid plexus and detectable protein deposition in the cortical parenchyma resulting in reversal of Ap deposition and improved clearance from the CNS (PMID: 24259049). To adapt the inventors' design for therapeutic application, the inventors replaced the eGFP reporter with human ApoE2 cDNA in constructs containing six different ependyma-enriched promoters (FIGS. 7 and 8). This library of six was further expanded by introduction of a short (133 bp) β-globin/IgG chimeric intron or a long (951 bp) chicken β actin/rabbit β-globin intron to test for intron-mediated enhancement of expression, totaling three variants for each gene.

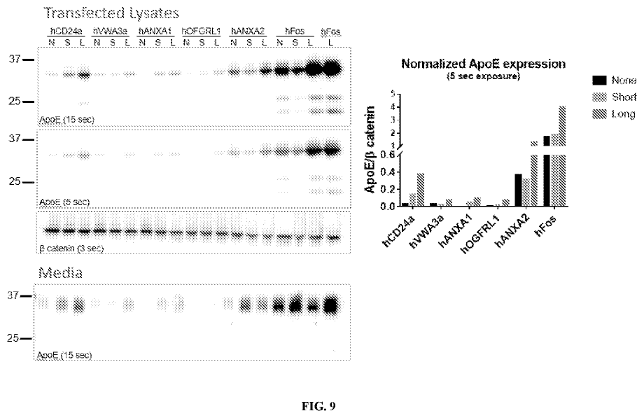

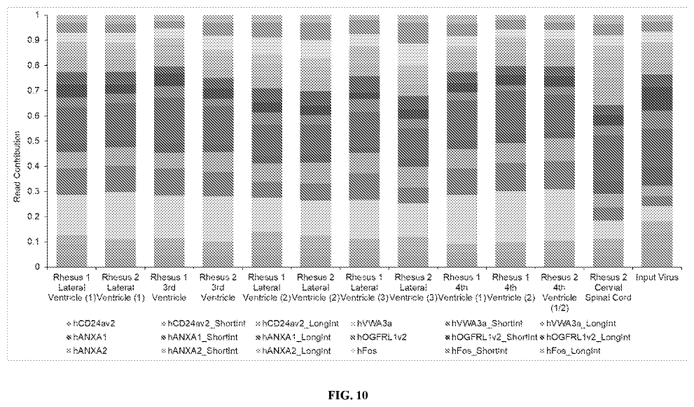

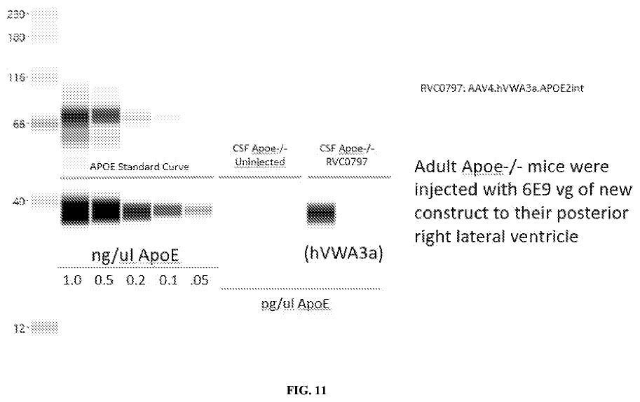

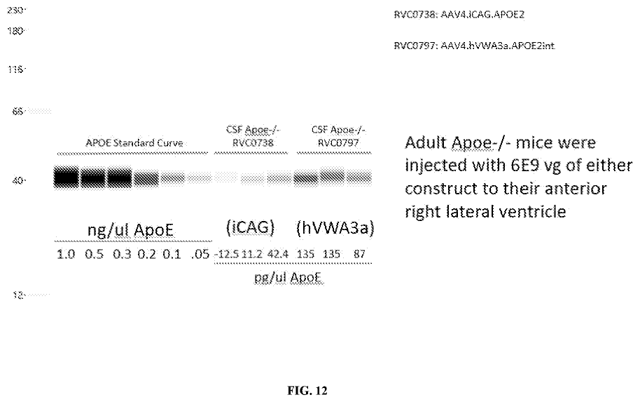

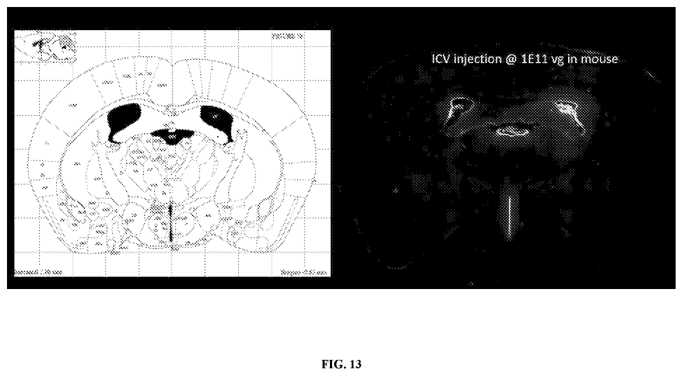

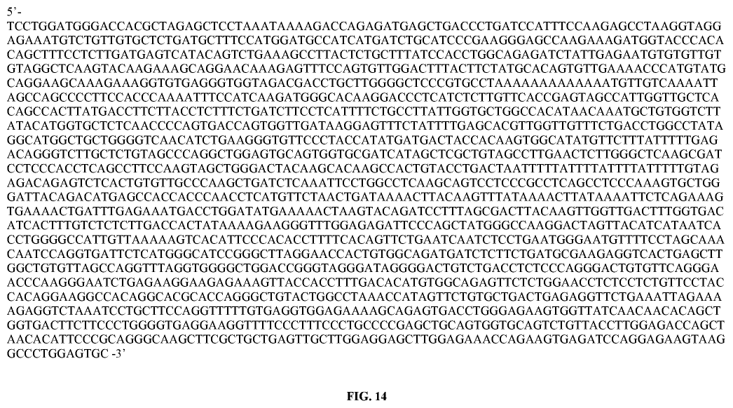

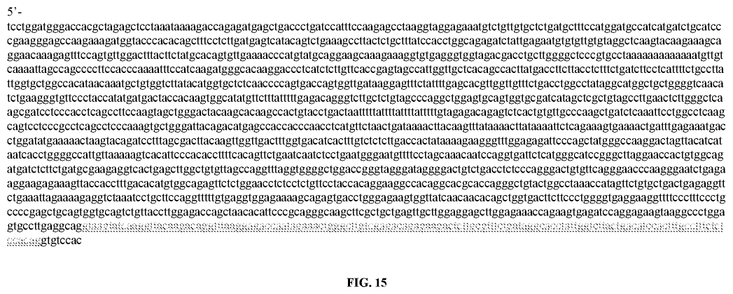

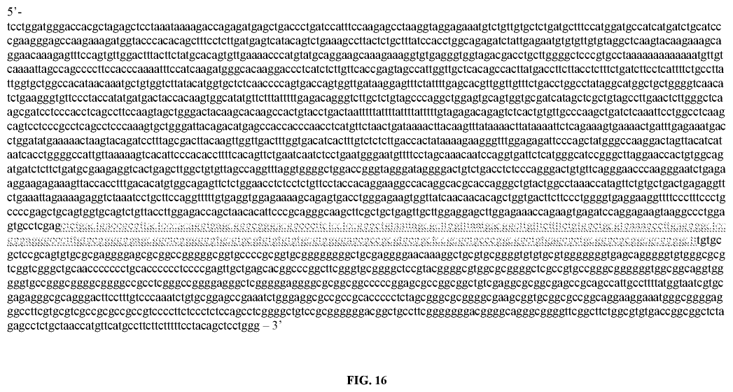

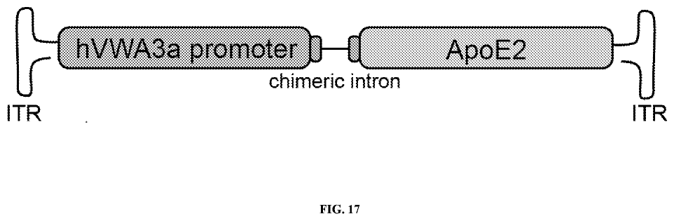

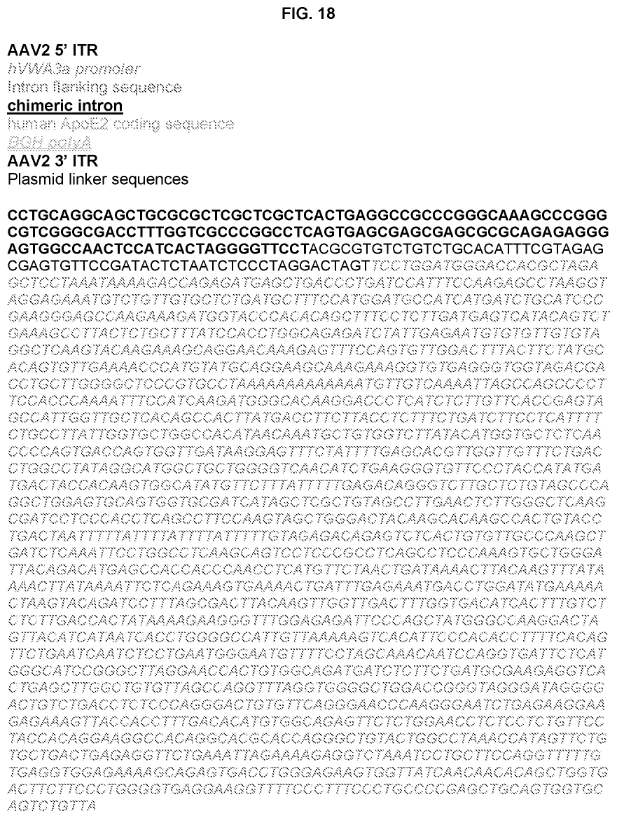

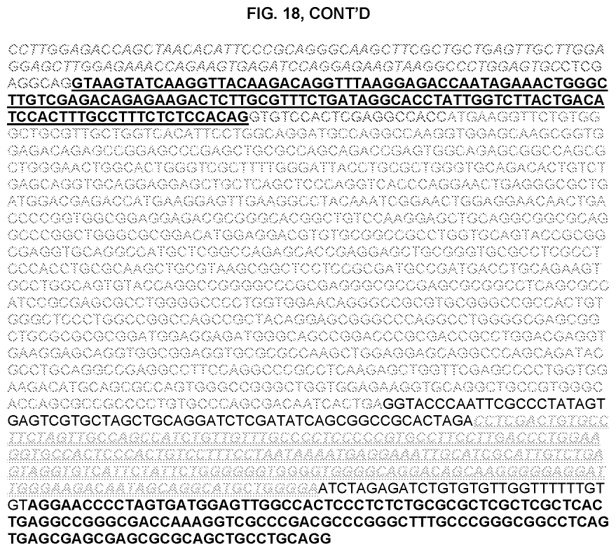

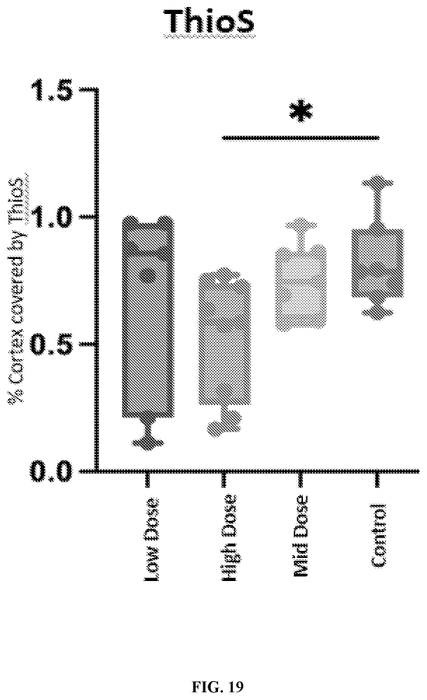

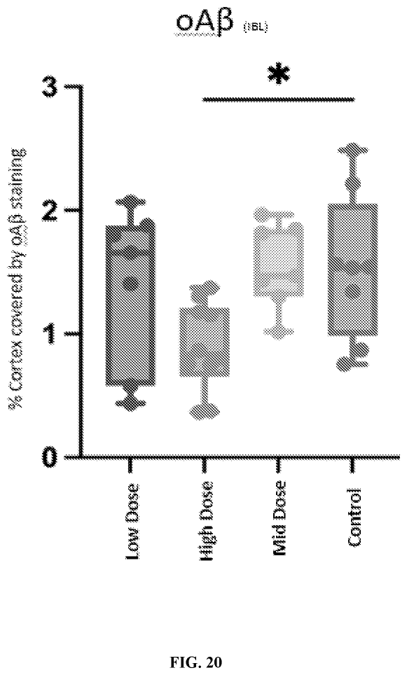

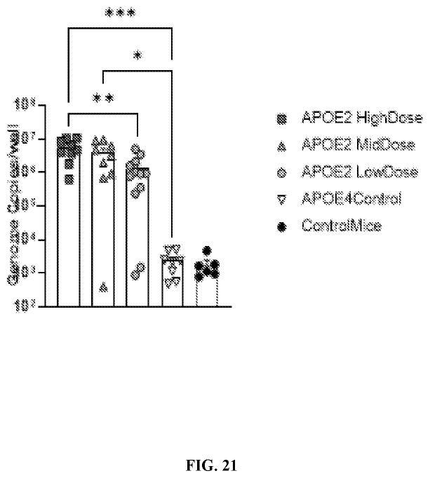

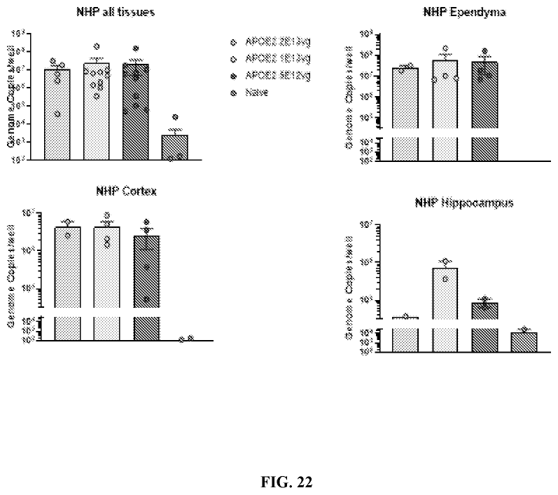

To measure intron splicing, individual plasmids were transfected into HEK293 cells and harvested at 24 hrs. RNA was isolated using Trizol, according to manufacturer's instructions and reverse transcribed using MultiScribe Reverse Transcriptase. Correct splicing was validated by amplifying across the intron-containing region in cDNA (C) vs plasmid DNA (D). Complete intron removal was observed in most transgenes except for the short intron from hANXA1 (FIG. 8). Protein output from the intron-containing transgenes was also measured in HEK293 cell lysates and the culture media. FIG. 9 contains a Western blot against APOE, a protein not readily detected endogenously in HEK293 cells. Consistent with intron-mediated enhancement, all variants showed increased protein output in the presence of the intron and to a greater extent with the long intron versus the short.

Measurement of intron-containing promoter activity in vivo. All 18 ApoE2 transgene variants (no intron, short, and long) were prepared as separate plasmids and pooled at equal molar ratios to generate a single AAV2 library. Virus was injected into the lateral ventricle of two adult rhesus macaques with a total dose of 2.8e13 vg per animal. After four weeks, ventricle margins were micro-dissected from 4 mm thick coronal slabs, and RNA was isolated using Trizol. cDNA amplicon sequencing of a product containing a unique 3-letter barcode in the 3′ UTR was used to assess relative promoter use in vivo (FIG. 10). Numbers in parentheses indicate isolation from comparable slabs in the larger indicated structure. All hVWA3a variants showed relative enrichment in vivo over the input library.

Confirmation that hVWA3a promoter can drive transgene expression in a mouse model. Adult APOE−/− (null) mice were injected with serotype AAV4 delivering APOE2 under the hVWA3a promoter to their right lateral ventricle. Ependyma tissue was microdissected and protein extracted for APOE2 quantification by automated Western blot technology (WES) compared to un-injected brain tissue.

Confirmation that hVWA3a promoter exhibits higher expression of APOE2 relative to the previous construct. APOE−/− (null) mice were injected with serotype AAV4 delivering APOE2 under either a ubiquitous CAG promoter or the hVWA3a promoter to their right lateral ventricle at equal doses. Protein was extracted from ependymal tissues microdissected from all animals and subjected to automated Western blot technology (WES). From the intensity of the bands, APOE2 driven by hVWA3a expressed higher amounts of APOE2 protein than the CAG promoter.

Biodistribution of novel capsid ERDRpAAV1 combined with hVWA3a restricts expression to ependymal cells in mouse models as visualized by eGFP transgene. Peptide modified AAV1 capsid with human ependymal specific promoter: ERDRpAAV1.hVWA3a.eGFP. Positive eGFP fluorescent signal is restricted to the ependymal cells lining the ventricles.

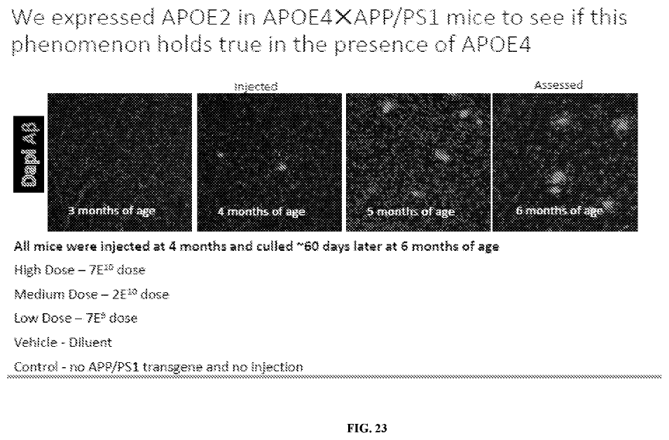

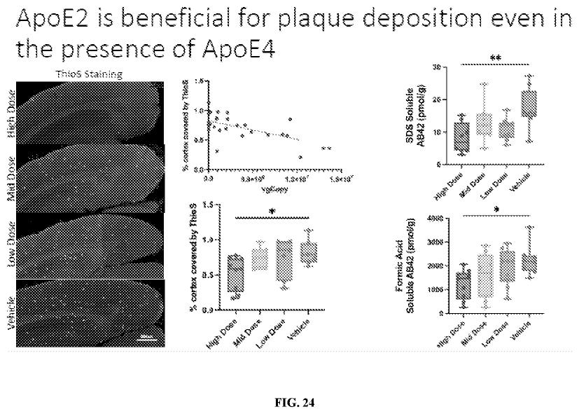

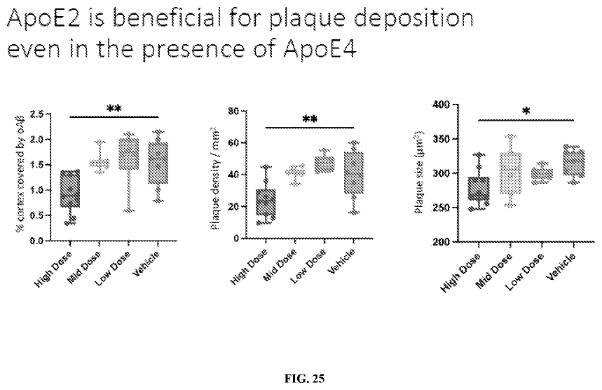

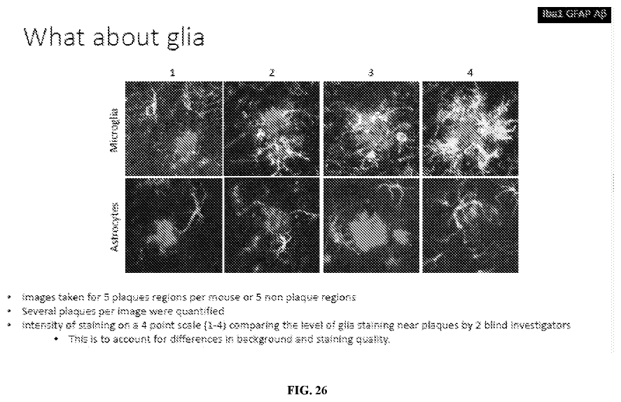

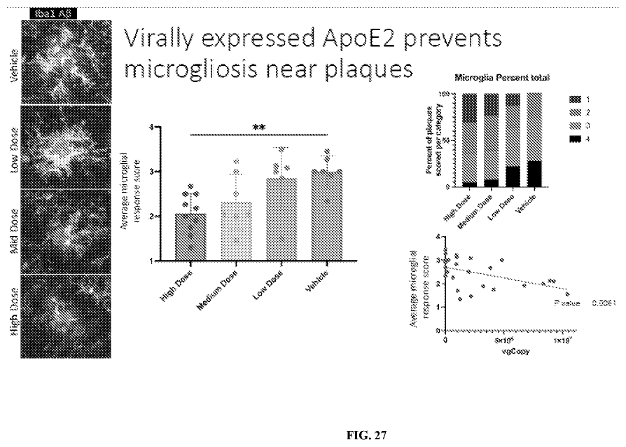

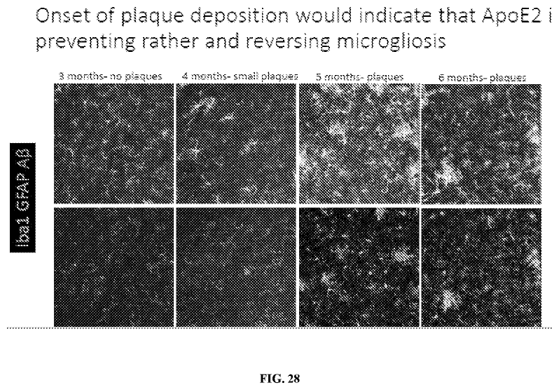

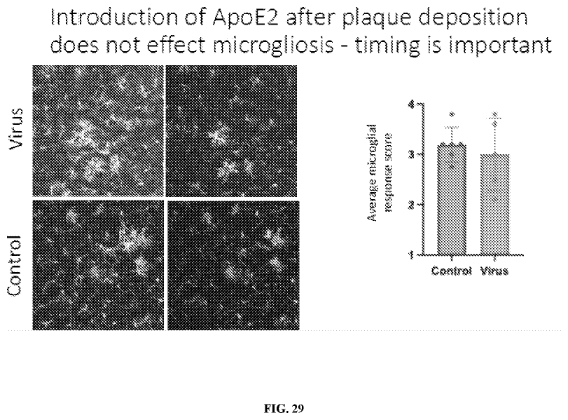

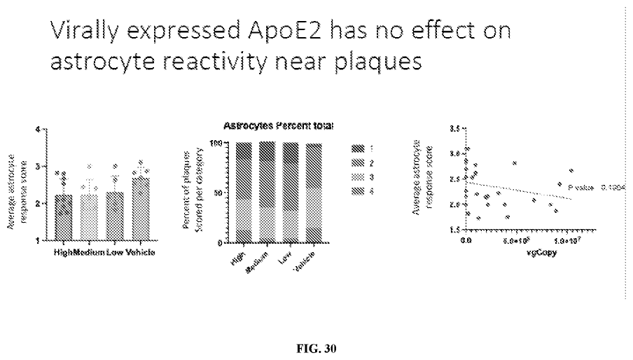

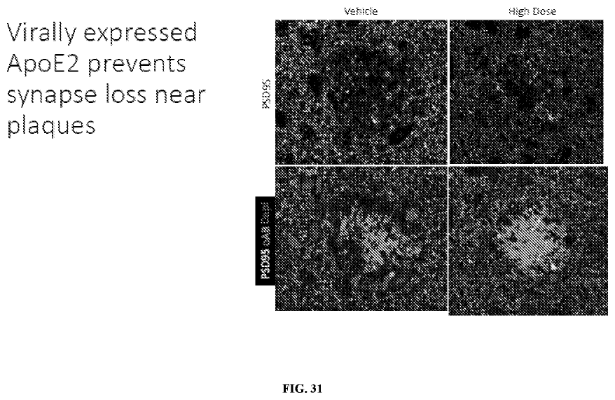

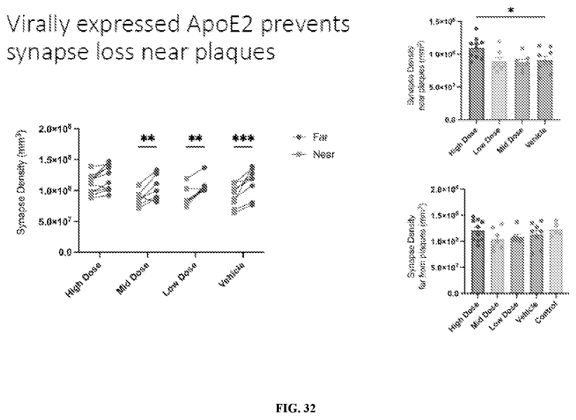

The introduction of ApoE2 via ependymal expression in this APP/PS1/ApoE4 mouse model reduced the size and density of cortical amyloid plaques and reduced the concentration of oligomeric Aβ in the brains of these animals. In addition, the inventors observed that the introduction of ApoE2 in this APP/PS1/ApoE4 mouse model dramatically reduces the activation of microglia near plaques in the cortex. This indicates that ApoEε4 might prime the microglia towards an inflammatory phenotype and that exogenous ApoE2 is able to interrupt or reduce this aberrant activation. The inventors also found that ApoE2 is able to prevent or lessen the loss of synapses that occurs in a halo around amyloid beta plaques in these mice. Together in concert this indicates that the effect of ApoE2 on the microglia might be protecting against the aberrant engulfment of synapses near plaques that likely occurs as a result of amyloid deposition. See FIGS. 23-34.

These results suggest that viral delivery of secreted ApoE2 into the ependyma ApoE4 carriers might be an effective therapeutic strategy to impact both the classical lesions of AD (eg plaque deposition and neurodegeneration) and the increased neuroinflammatory profile observed in sporadic AD.

### Example 2

Methods. Study design. The inventors performed intracerebroventricular (ICV) infusion with a novel AAV vector expressing the APOE2 variant within the cerebroventricular space of an AD transgenic mouse model. Mice were injected with either AAV or a vehicle control for a 2-month period. Using immunohistochemistry (IHC) and enzyme-linked immunosorbant assays (ELISAs) the inventors evaluated the impact of human APOE2 on amyloid deposition. Mice were randomly assigned to treatment groups. The nature of the injected vector was kept blinded until statistical analysis. They estimate needing 8 animals (4 of each sex) per condition for these studies. This will provide a power of >0.8 to see a correction of 30% of the baseline phenotype as compared with based on previous data (Hudry et al., 2013).

Animals. APP/SP1(Radde et al., 2006) mice express human mutant APP KM670/671NL and PSEN1 L166P under the Thy1 promotor, which leads to a severe phenotype characterized by amyloid deposition at 3-4 months of age. The APOE targeted replacement expresses human APOE4(Huynh et al., 2019) in the mouse model under the control of the murine APOE promoter. These animals were back-crossed until the APP/PS1 transgene was expressed alongside two copies of human APOE4 in place of mouse apoe (APPPS1/APOE4). The inventors exposed 4-month-old APOE4-TR/APP/PS1 mice to either a high (7E10 vg, n=14), medium (2E10 vg, n=11), or low (7E9 vg, n=11) dose of AAVert-APOE2 or a vehicle control (n=10) for 2 months. In addition, a cohort of APOE KO (Jackson labs) mice on a C57BL/6 background was included as a comparison measure for the levels of Af within the tissue and CSF. Experiments were performed in accordance with the National Institutes of Health (NIH) and institutional guidelines and both sexes were used. Due to the small size of the mouse brain not all animals were used in every analysis and n is indicated by number of points shown. Open circles indicate females and closed indicate males.

Viral vector construction and production. Research grade production of AAV-based viral vectors were manufactured by the Children's Hospital of Philadelphia Research Vector Core (RVC) by transient triple transfection of adherent human embryonic kidney epithelial cells (HEK293) from a certified Working Cell Bank (WCB). Cells were expanded in tissue culture flasks and roller bottles prior to transfection. Testing of research product was performed in-house by RVC QC and test methods, procedures and results are reported on a Certificate of Analysis (CoA) for each lot.

Stereotactic intracerebroventricular injections. AAV intracerebro-ventricular injections were performed as described previously (14, 30). Animals were anesthetized (O₂/Isoflurane 0.2%) and positioned on a stereotactic frame (David Kopf Instruments). Injections were performed in each lateral ventricle with 5.25 μl of viral preparation using a 33-gauge needle attached to a 10-1 Hamilton syringe (Hamilton Medical) at 0.20 μl/min. Stereotactic coordinates were calculated from bregma (anteroposterior+0.3 mm, mediolateral±1 mm, and dorsoventral−2 mm).

Western blot. Mouse cortical tissue was homogenized in 10 volumes by weight of ice-cold TBS with protease and phosphatase inhibitors using a hand-held electric homogenizer. The homogenate was then spun at 10 000 g for 10 min and the supernatant (TBS-soluble fraction) was collected for western blot. Protein concentration was determined using a BCA assay. Total protein (5-10 g) was loaded and separated by 4-12% NuPAGE gels in MES buffer, proteins were then separated by weight for 2 h at 120 V. Proteins were electrotransferred onto nitrocellulose membrane at 30 V for 1.5 h using the XCell II™ Blot Module system in tris-glycine transfer buffer. Membranes were incubated in blocking buffer (Li-Cor Biosciences) diluted 1:1 TBS for 1 h to reduce background staining. Membranes were then incubated with primary antibodies; rb anti-APOE (Novus biologicals, NBP1-31123), and ms anti-GAPDH (Millipore MAB374) diluted in blocking buffer with added 0.1% Tween-20 overnight at room temperature while shaking. Membranes were then washed and incubated with the appropriate 680 and 800 IR dye secondary antibodies (Li-Cor Biosciences). The membranes were imaged using Odyssey infrared imaging system and analyzed using Odyssey software.

DNA and RNA extraction and analysis. Genomic DNA was extracted from brain tissue using QIAamp DNA Mini Kit (Qiagen) as per manufacturer's protocol. Samples were run on BioRad CFX384 Real Time System C1000 Touch using BioRad CFX Manager 3.1 software. Total genome copies were quantified against a 6-point standard curve was generated using linearized plasmid containing the construct. Primer/probes (designed against a non-coding region in the construct) was used with TaqMan® Master Mix (Applied Biosystems).

Total RNA was extracted from brain tissue using TRIzol (Ambion by Life Technologies) as per manufacturer's protocol. RNA (1 μg) was treated with DNase I, RNase-free (ThennoScientific) as per manufacturer's protocol. Complementary DNA was generated using the High Capacity cDNA Reverse Transcription Kit (Life Technologies). Samples were run on BioRad CFX384 Real Time System C1000 Touch using BioRad CFX Manager 3.1 software. APOE levels were quantified by designed primer/probes to be used with TaqMan® Master Mix (Applied Biosystems). Exogenous mRNA levels of transgene expression human APOE (Hs00171168_m1) commercial TaqMan® primer/probe set (Applied Biosystems). Endogenous mouse Beta-Actin (Mm02619580_g1) was used as a reference gene to normalize expression across samples.

ELISA quantification. The concentrations of A040 and A042 were determined by BNT-77/BA-27 (for AQ40) and BNT-77/BC-05 (for AB42) sandwich ELISA (Wako) according to the manufacturer's instructions. A040 and A042 concentrations were measured in TBS, SDS-soluble, and SDS-insoluble fractions for each mouse. Sections of mouse brain were homogenized in 10 volumes (w/v) of TBS buffer with a cOmplete protease inhibitor cocktail (Roche), and centrifuged at 1000,000×g for 30 min at 4° C. The supernatant was collected and set aside as the TBS-soluble fraction. The pellet was then homogenized in 10 volumes (w/v) of TBS buffer containing 2% SDS, incubated at 37° C. for 30 min and then centrifuged at 100,000×g for 30 min at 20° C. The SDS-insoluble pellet was dissolved in 500 μl of 70% formic acid and sonicated on ice at 10% power in 1 minute and 30 second intervals until completely dissolved, and then centrifuged at 100,000×g for 30 min at 4° C. The formic acid-soluble supernatant was desiccated by Speed-Vac and then resuspended 1 volume (w/v) of dimethyl sulfoxide (DMSO). The DMSO-soluble fraction was used as a SDS-insoluble fraction (adapted from Hashimoto et al 2020) (Hashimoto et al., 2020).

RNAscope. The drop fixed hemisphere of the APOE KO mice was sectioned to 30 μm on a freezing ultramicrotome. Three mice per experimental condition (sham injection vs AAV injected) were stained for APOE mRNA by RNAscope. RNAscope experiments were performed using the Manual Fluorescent Multiplex kit v2 (Advanced Cell Diagnostics) following manufacturer's recommendations with minor adjustments. Briefly for each mouse a several sections were baked onto a superfrost slide for use in ΛPOE mRNA quantification. Following target retrieval and protease digestion, probe hybridization was carried out at 40° C. for 2 h with hs-APOE (433091), 3-plex Positive Control Probe_Mm (320881) and Negative Control Probe-DapB (310043). After amplification steps to obtain the RNAscope signals, the signal was developed using TSA-cy3 (Perkin Elmer FP1170). Sections were counterstained with 1:1000 dapi, and mounted using immunomount and scanned using an Olympus VS120-S6-W virtual slide microscope, at a magnification of ×10.

Immunohistological analysis. Mice were euthanized by isoflurane inhalation. One cerebral hemisphere was fixed in 4% paraformaldehyde and 15% glycerol in PBS and switched to 30% glycerol in PBS 48 hours later. The remaining hemisphere was snap-frozen for biochemical analysis. Drop fixed hemispheres were processed by neuroscience associates. 40 hemispheres were embedded in a gelatin block and sectioned to 30 μm. Sections were permeabilized in 0.5% Triton-X for 15 min before being blocked in 0.1% Triton-X and 5% normal goat serum for 1 h at room temperature. Incubation with primary antibodies was done overnight at 4° C. in 0.05% Triton-X and 2.5% normal goat serum. Sections were then washed in TBS and appropriate secondary antibodies were diluted 1:500 in 0.05% Triton-X and 2.5% normal goat serum in TBS at room temperature. Sections were incubated with 1:1000 dapi in TBS for 10 minutes at room temp, washed, and mounted using immunomount.

Plaque quantification. Every 10^{th} section was stained as described above using rabbit anti Abeta (1:500, IBL, CAT #18584) for amyloid beta. Amyloid dense core plaques were labeled by 0.05% Thio-S(Sigma-Aldrich) in 50% ethanol before mounting. Sections were mounted and scanned using a nanozoomer microscope at 40×. Sections were quantified using qupath (Bankhead et al., 2017). For each section cortical areas were selected, and plaques were identified using an object classifier and plaque coverage area was assessed as a percent of the cortical area measured. For plaque size and number, the same sized area was selected in the cortex of each animal and plaques were identified using an object classifier.

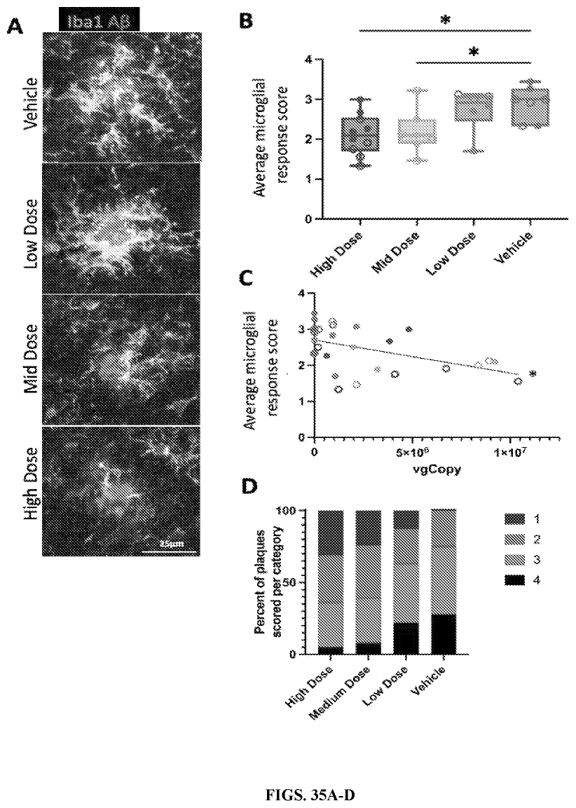

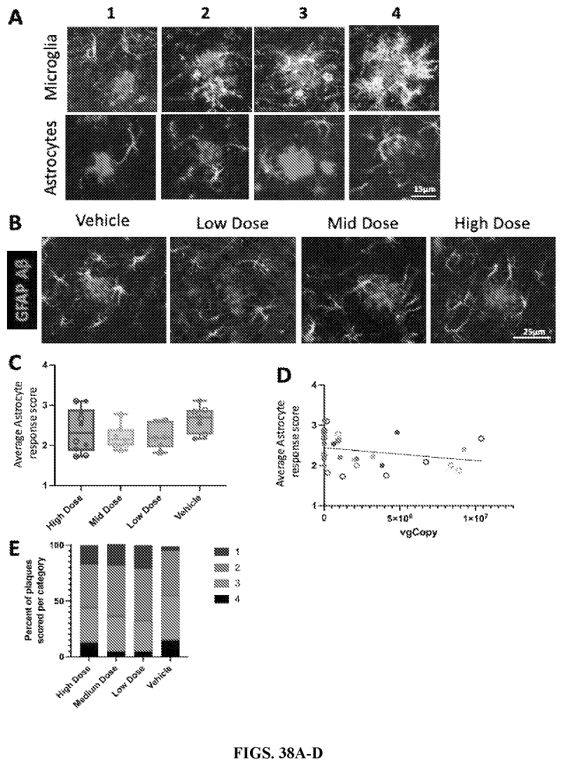

Glial assessment. Several sections were stained as described above. Primary antibodies were 1:1000 biotinylated Ms anti Abeta 82E1 (IBL 10326), 1:500 GFAP-488 (Millipore MAB 3402X), and 1:500 rabbit anti-IBA1 (wako 019-19741). Secondaries were Streptavidin Alexa Fluor 568 (Invitrogen S11226) and Donkey anti-rabbit 647 (A-31573). 5 cortical plaque containing areas were imaged at random from the somatosensory cortex using an Olympus FV3000 confocal laser scanning microscope at 40×. Z stacks were generated and each plaque was assessed on a 4 point scale (FIG. 38A) for the level of glial reactivity by 2 blind investigators. All plaques for an individual mouse were averaged together to generate the graphs in FIGS. 35B-C and FIGS. 38C-D while all plaques from a given experimental group were assessed for FIG. 35D and FIG. 38E.

Synapse quantification. Several sections were stained as described above. Primary antibodies were using 1:500 rabbit anti Abeta 1:500 (IBL, CAT #18584) and 1:500 goat anti PSD95 (abcam ab12093). Secondaries were Donkey anti-goat 488 (Invitrogen A-11078) and Donkey anti-rabbit 594 (A A-21207). 5 cortical plaque containing areas were imaged at random from the somatosensory cortex using an Olympus FV3000 confocal laser scanning microscope at 60× using an oil dipping objective. 5 μm of Z stack was imaged at a slice size of 0.56 m. Images were processed using custom Image J and Matlab Macros similar to Jackson et al. (Jackson et al., 2019). In brief 10 m×10 m crops were taken from areas within 15 μm of the plaque halo or greater than 40 m from the plaque halo. Cellular debris and dapi were avoided. Crops thresholded using custom image J macros and synapses quantified using custom matlab macros. All crops were averaged together to find a density near and far from plaques for each mouse.

Neurite quantification. Several sections were stained as described above. Primary antibodies were using 1:500 rabbit anti Abeta 1:500 (IBL, CAT #18584) and 1:500 mouse anti SMI312 (Biolegend 837904). Secondaries were Donkey anti-rabbit 488 (Invitrogen A-21206) and Donkey anti-mouse 594 (A-21203). 5 cortical plaque containing areas were imaged at random from the somatosensory cortex using an Olympus FV3000 confocal laser scanning microscope at 60× using an oil dipping objective. 20 μm of Z stack was imaged at a slice size of 1 μm. Images were quantified using Image J by a blind experiment who counted number of dystrophies per plaque and also quantified plaque area. In images where more than one plaque was present the largest plaque was quantified.

Statistical analyses. Statistical analyses were performed with the Prism software. One-way ANOVA was used to analyze all between group analysis followed by Dunnett's multiple comparisons test between each group and the vehicle control. Simple linear regression analysis was used to assess the correlation between factors and the number of viral genome copies with p values representing if the slop was significantly non-0. Statistics were performed where each mouse was a single data point. Samples were blinded for each analysis.

Results. Intraventricular injection of AAVert-APOE2 leads to sustained production of APOE2 in the brain in a dose dependent manner. APOE is produced predominantly by astrocytes and microglia cells within the CNS and once produced it is secreted after which it can diffuse throughout the parenchyma. Previously, the inventors showed that APOE2 produced by the cells of the ependymal lining the ventricle can diffuse as far as the cortex where it effects plaques and plaque associated damage (Hudry et al., 2013).

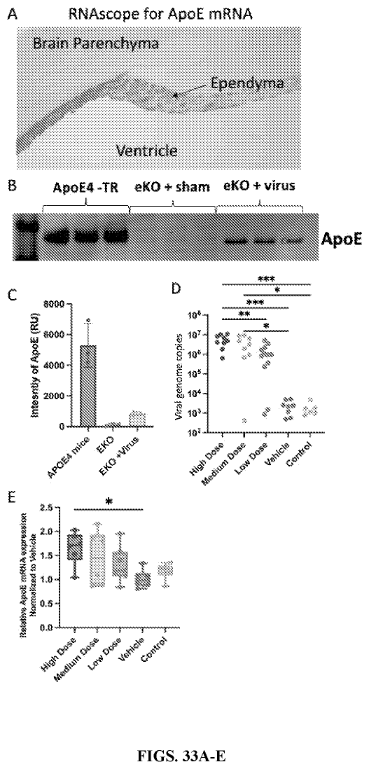

The inventors performed a single intracerebroventricular (ICV) injection of a novel ependymal restricted AAV capsid (AAVert) expressing APOE2 into four-month-old APOE KO mice. which were sacrificed 2 months later. Injection with the high dose (7E¹⁰ genome units (vg)) of virus into Apoe KO mice resulted in robust expression of APOE2 mRNA in the ependymal cell lining of the ventricle detected using RNAscope for human APOE (FIG. 1A). Virus driven APOE2 protein was also detected in a TBS extraction of the cortex by western blots and is shown to be 10% of normal APOE (FIGS. 33B-C).

The inventors then went on to inject AAV carrying APOE2 into four-month-old APPPS1/APOE4 animals which were culled two months later. They injected animals at three different doses: Low-7E⁹ vg, Mid-2E¹⁰ vg, and High-7E¹⁰ vg, as well as a vehicle control group. DNA extraction from the hindbrain followed by qPCR showed a dose dependent effect of uptake as well as three animals that showed no uptake (FIG. 33D). The number of viral genome copies correlates with an increase in the amount of human APOE mRNA which is ˜50% higher in the high dose animals than endogenous levels observed in the vehicle treated animals (FIG. 33E). Together these data indicate that a single ICV injection induces APOE2 expression in the ependymal cells in a dose dependent manner.

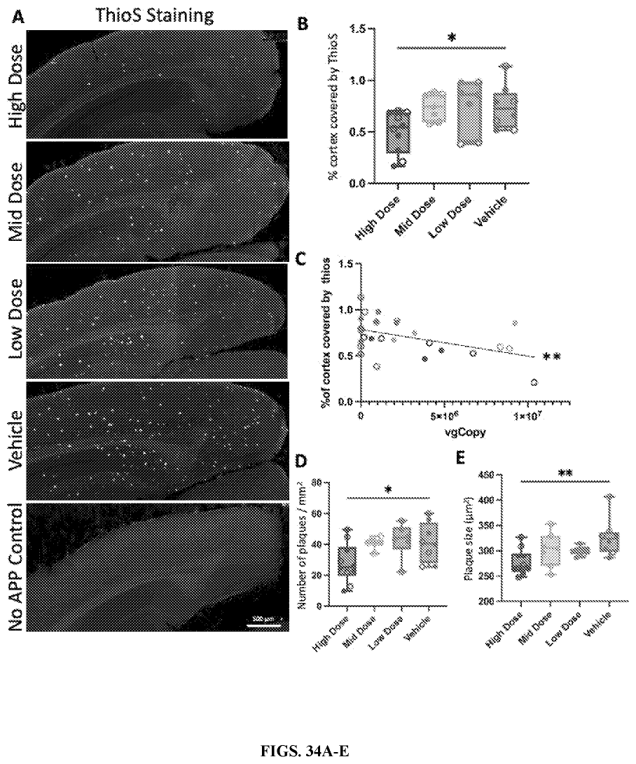

Expression of APOE2 has a dose dependent effect on the level of AB plaque deposition. The APP/PS1/APOE4 model is a relatively aggressive model of amyloidosis. At four months of age APPPS1/APOE4 animals show modest plaque deposition and by six months of age plaque deposition is well established across the cortex. The inventors therefore injected at 4 months and sacrificed at 6 months. Using ThioS as a marker for dense core amyloid plaques they show a dose dependent effect of APOE2 on plaque deposition (FIGS. 34A-C). The high dose animals show a ˜33% reduction in the percent of the cortex covered by ThioS positive staining as compared with the vehicle treated animals (FIG. 34B). The dose dependent effects of APOE2 on plaques is clear when plaque burden is compared with the number of viral genome copies extracted from the hind brain (FIG. 34C). Staining using an anti-oligomeric Aβ (oAβ) antibody showed a similar trend in both the group (Supplemental FIG. 1A) and the individual level (Supplemental FIG. 1B).

The observed reduction in percent cortex covered is accompanied by a significant reduction in plaque density (FIG. 34D) where high dose animals showed a reduction in number of plaques per mm² when compared with vehicle treated animals and an even more significant reduction in the size of Aβ plaques (FIG. 34E).

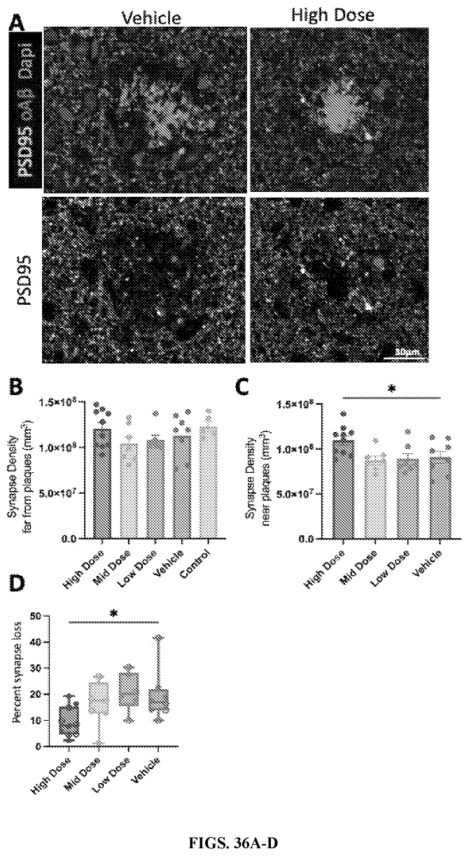

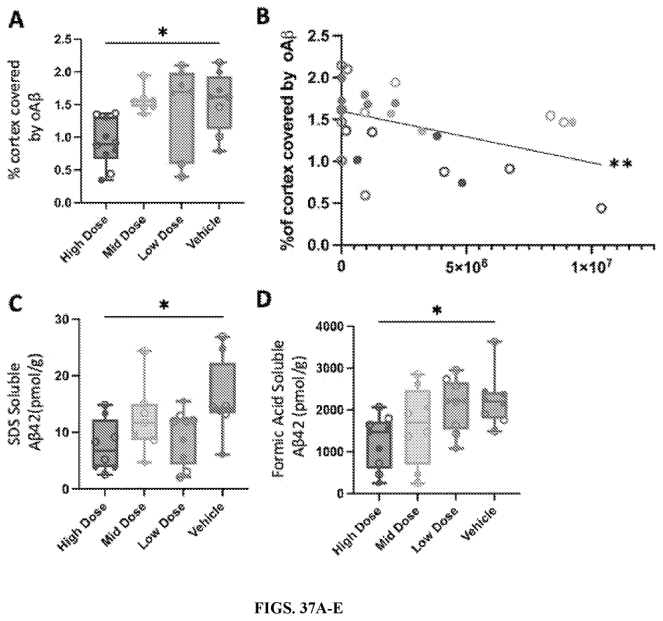

Biochemical measures of amyloid align with the imaging measures. The concentrations of Aβ42 peptides measured from formic acid and SDS soluble extracts of mouse brain mimicked the changes observed histologically such that the high dose animals showed a ˜50% reduction in the amount of both SDS (FIG. 36C) and formic acid (Supplemental FIG. 1D) soluble Aβ42.

APOE4 has been shown to impair clearance and promote aggregation of Aβ while APOE2 has been shown to have the opposite effect. The data presented here are consistent with an increased efflux of Aβ peptides across the BBB in the presence of APOE2 which results in a reduction of plaques.

Expression of APOE2 has a dose dependent effect on Plaque related neuroinflammation. APOE4 mice have been shown to have a more aggressive neuroinflammatory response to plaques when compared with APOE2 or APOE3 mice. This reflects human disease as data has shown that APOE4 carriers have a more inflammatory phenotype (Serrano-Pozo, Li, et al., 2021). To see if the addition of APOE2 could attenuate the effect of plaques on the local astrocytes and microglia the inventors performed IHC for AD, Iba1 (microglia) (FIG. 35A) and GFAP (astrocytes in FIG. 38B). They assessed the level of glial reactivity on a 4-point scale where 1 is non-reactive and 4 is very reactive (FIG. 38A). The images were assessed by 2 blinded investigators with a correlation R² of 0.89. The inventors chose to assess the area immediately surrounding individual plaques rather than the entire cortex as microglial activation in these mice is closely associated with plaques and thus high dose animals with less plaques would have lower global levels of active microglia. By utilizing this method, the inventors are able to look at microglial reactivity uncoupled in part from gross plaque density.

The inventors found that an obvious, statistically significant reduction in microglial reactivity in the high and mid dose groups as compared with the vehicle treated mice (FIG. 35B) and this attenuation shows significant correlation with the number of viral genome copies in individual animals (FIG. 35C). This reduction appears to be driven by an increase in the number of plaques that have not caused a strong microglial reaction (score of 1) in the high and mid dose animals and a reduction in plaques that have a score of 4 as compared with vehicle treated (FIG. 3D). In contrast astrocyte reactivity around plaques is unaffected by APOE2 levels or expression (FIGS. 38A-D) and is equally elevated around plaques in all groups.

APOE2 exposure modulates synaptic loss around amyloid deposits. Synapse loss is known to correlate with cognitive impairment and has been shown to occur near plaques in human patients (Koffie et al., 2012) as well as in this mouse model (Hudry et al., 2013). The inventors have previously shown that APOE4 in both carriers and mice is associated with higher amount of synaptic loss near plaques compared with APOE3 mice or carriers. As APOE4 has been shown to fail to protect against synapse loss, they tested the hypothesis that the addition of APOE2 would be able to protect synapse integrity in this model.

Post-synaptic densities (PSD95) were identified using immunohistochemistry and imaged by confocal microscopy (FIG. 38A). Synapse loss in this model has been shown to occur most prominently within 15 μm of the plaque edge Hudry et al. 2013; Koffie et al., 2012) therefore crops were taken from this area analyzed as were crops taken more than 40 μm from a plaque edge. Synapse density far from plaques did not differ among groups (FIG. 38B); however, animals injected with the highest dose showed an increased level of synapses near plaques compared with vehicle treated animals (FIG. 38C), restoring to near normal synaptic density. Percent synapse loss was calculated by comparing the synaptic density near plaques with synaptic density far from plaques in the same animal (FIG. 38D). Vehicle treated animals show twice as much synapse loss as high dose animals with 70% of high dose animals showing less than 10% loss near plaques as compared with the other groups where all but one animal showed more than 10% loss.

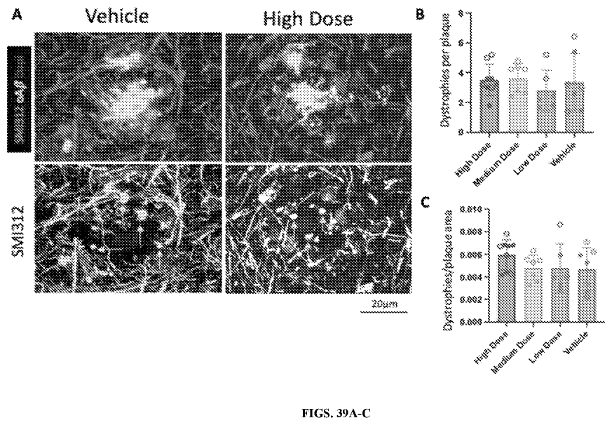

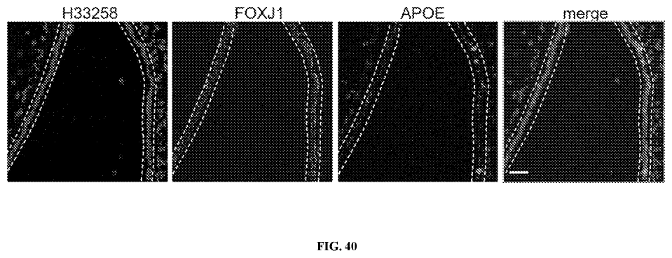

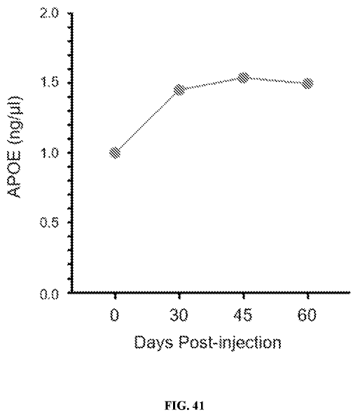

The inventors also evaluated the number of dystrophic neurites associated with amyloid deposits by staining for the axonal marker SMI312 alongside an oligomeric Aβ antibody (FIG. 39A). They found no difference in the number of neuritic dystrophies among the groups (FIGS. 39B-C).

Discussion. The strong genetic and experimental link between APOE genotype and AD has long made APOE a subject of interest when considering AD risk modifiers or therapeutics (Serrano-Pozo, Das, et al., 2021). However, the practical issues of distributing gene product throughout the brain is a barrier to using gene therapy in disease that effect large volumes of brain tissue like AD. This is especially true in larger organisms such as non-human primates or humans. This current study has tested an approach to overcome this barrier: expression of secreted proteins via transduction of the ependyma and related structures, allowing secreted protein to diffuse throughout the cortical mantle. The inventors applied this approach to expressing APOE2 in an APOE4/APP/PS1 model of Alzheimer pathology, and show that, in achievable doses, expression of APOE2 can positively impact plaque deposition, neuroinflammation, and neurodegeneration within 8 weeks of treatment.

Importantly, this improvement is observed in the setting of established disease (i.e., plaque deposition had already begun), modeling from a neuropathological perspective the changes that would occur in patients with established early Alzheimer disease. The results presented here suggest that the impact of APOE in AD is continuous throughout disease progression, rather than a consequence of developmental or midlife changes associated with APOE4 (Evans et al., 2020) and that manipulating APOE even after AD is established can alter the course of the disease.

The inventors tested 3 doses of virus to determine the minimum effective amount of AAV and APOE2 needed to see an effect in this very aggressive model of amyloidosis (FIG. 2). Due to the work of others (Castellano et al., 2011; Hashimoto et al., 2012; Serrano-Pozo, Das, et al., 2021), the inventors hypothesize that the main effect of APOE2 on the hallmark AD pathology of Aβ plaques is due to an increase in the clearance of Aβ across the BBB and well as a decrease in aggregation. This study shows that APOE2 production effect on both dense core fibrillar plaques and biochemical measures of Aβ, consistent with this conclusion.

Neuroinflammation and AD have long been linked in part due to the number of microglial genes that are risk factors for AD and also due to the marked increase in micro- and astrogliosis in brains from both AD cases and mouse models (Leng & Edison, 2021). In recent years the effects of APOE genotype on neuroinflammation have been studied (Hong et al., 2016). Microglia produce APOE under basal conditions but production is dramatically increased in these cells when inflamed. This observation has led to the hypothesis that microglial produced APOE4 has a gain of toxic function effect in causing or perpetuating a feed forward loop (Krasemann et al., 2017), raising the possibility that the effect of APOE is cell autonomous. The inventors show that exogenous APOE2 produced by the ependymal cells dampens microglial activation near plaques in mice (FIG. 3), which is not consistent with this hypothesis and indicates that at least some of the effect of APOE on microglia are due to APOE produced in other cell types.

APOE4 has been associated with more severe synapse loss near plaques in AD (Hudry et al., 2013; Koffie et al., 2012). In mice with the highest dose of AAV, the inventors see a reduction in the amount of synapse loss indicating that APOE2 could prevent this neurodegenerative phenotype. They have previously shown that APOE and oligomeric oAβ colocalize at synapses, and that APOE4 is more efficient at delivering oAβ to the synapse(Koffie et al., 2012). This synaptoprotective effect of APOE2 could be due to a number of mechanisms that do not preclude one another. APOE2 could help reduce bioactive oAβ present at the synapses, the effect of APOE2 on microglia could cause reduced levels of synaptic pruning due to reactive microglia, and increased clearance of oAβ could reduce the amount of oAβ in the halo of plaques. It is likely a combination of these effects that lead to a lack of toxic oAβ at the synapse reducing both microglial pruning and the synaptotoxic effect of oAβ.

The data presented here highlight the utility of an ependymal restricted AAV for the expression of secreted proteins which, once secreted into the neuropil and CSF, can diffuse throughout the brain parenchyma. This approach demonstrates the practical possibility of using this method of gene therapy to treat the entire brain, something that would be of great significance in lysosomal storage diseases, and to diseases such as progranulin loss of function in progranulin mutation linked frontotemporal dementia. The inventors envision ultimately the ability to introduce into the brain a variety of potential therapeutics, from single chain antibody production to expression of other bioactive molecules. The current study establishes in principle 2 things; APOE2 protein as a therapeutic, and an AAV platform for therapeutics where either blood brain barrier issues or difficulties with peripheral expression (including clearance) currently preclude use for CNS disorders.

In conclusion, the data presented here suggest that gene therapy introduced APOE2 has a protective function that parallels well established phenotypes in human patients who have inherited the E2 or E4 alleles. In this model even modest levels of APOE2 expression impacts Aβ deposits, attenuates neuroinflammation, and supports synaptic systems. This speaks to APOE modulation being an important possibility for disease altering therapeutics in patients with Alzheimer disease.

All of the methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

The following references, to the extent that they provide exemplary procedural or other details supplementary to those set forth herein, are specifically incorporated herein by reference.
U.S. Patent Publication 20110059502
U.S. Pat. No. 6,217,552
Gonzalez et al., BMC Neurosci 10.1186/1471-2202-12-4 (2011)
Lazorthes et al. Advances in Drug Delivery Systems and Applications in Neurosurgery, 18:143-192(1991)
Ommaya et al., Cancer Drug Delivery, 1:169-179 (1984)
Ommaya, Lancet 2: 983-84 (1963)

1. A method of expressing a therapeutic transgene in ependymal tissue of a subject, comprising administering to the subject a modified adeno-associated virus (AAV) encoding a therapeutic transgene under the control of a promoter selected from the group consisting of SEQ ID NO: 1, SEQ ID NO: 2, SEQ ID NO: 3, or a promoter having at least about 80% sequence identity therewith.

2. The method of claim 1, wherein the promoter is SEQ ID NO: 1 or a promoter having at least about 85%, 90%, 95%, 96%, 97%, 98% or 99% sequence identity therewith.

3. The method of claim 1, wherein the promoter is SEQ ID NO: 2 or a promoter having at least about 85%, 90%, 95%, 96%, 97%, 98% or 99% sequence identity therewith.

4. The method of claim 1, wherein the promoter is SEQ ID NO: 3 or a promoter having at least about 85% t, 90%, 95%, 96%, 97%, 98% or 99% sequence identity therewith.

5. The method of claim 1, wherein the modified AAV comprises a modified capsid protein.

6. The method of claim 5, wherein the modified capsid protein comprises a targeting peptide, wherein the targeting peptide is three to ten amino acids in length, such as seven amino acids in length.

7. The method of claim 5, wherein the modified AAV capsid protein is a modified AAV1 capsid protein, a modified AAV2 capsid protein, or a modified AAV9 capsid protein.

8. The method of claim 7, wherein the modified AAV capsid protein is derived from an AAV1 capsid protein, wherein the targeting peptide is inserted after residue 590 of the AAV1 capsid protein.

9. The method of claim 8, wherein the targeting peptide is flanked by linker sequences, wherein the linker sequences on each side of the targeting peptides are two or three amino acids long.

10. The method of claim 9, wherein the linker sequences are SSA on the N-terminal side of the targeting peptide and AS on the C-terminal side of the targeting peptide.

11. The method of claim 7, wherein the modified AAV capsid protein is derived from an AAV2 capsid protein, wherein the targeting peptide is inserted after residue 587 of the AAV2 capsid protein.

12. The method of claim 11, wherein the targeting peptide is flanked by linker sequences, wherein the linker sequences on each side of the targeting peptides are two or three amino acids long.

13. The method of claim 12, wherein the linker sequences are AAA on the N-terminal side of the targeting peptide and AA on the C-terminal side of the targeting peptide.

14. The method of claim 7, wherein the modified AAV capsid protein is derived from an AAV9 capsid protein, wherein the targeting peptide is inserted after residue 588 of the AAV9 capsid protein.

15. The method of claim 14, wherein the targeting peptide is flanked by linker sequences, wherein the linker sequences on each side of the targeting peptides are two or three amino acids long.

16. The method of claim 15, wherein the linker sequences are AAA on the N-terminal side of the targeting peptide and AS on the C-terminal side of the targeting peptide.

17. The method of claim 1, wherein the therapeutic transgene is an siRNA, shRNA, miRNA, non-coding RNA, lncRNA, therapeutic protein, or CRISPR system.

18. The method of a claim 1, wherein the therapeutic transgene is ApoE2 and the subject suffers from or is at an increased risk of developing Alzheimer's Disease as compared to the populational average.

19. The method of claim 1, wherein the administration is direct intracerebroventricular or intraparenchymal injection.

20. The method of claim 1, wherein the modified AAV is administered more than once.

21. The method of claim 20, wherein the modified AAV is administered 2, 3, 4, 5, 6, 7, 8, 9, 10 or more times.

22. The method of claim 20, wherein the modified AAV is administered monthly, every other month, every three months, every four months, every six months or annually.

23. The method of claim 1, further comprising providing a non-AAV therapy to said subject.

24. The method of claim 1, wherein a plurality of viral particles are administered.

25. The method of claim 24, wherein the virus is administered at a dose of about 1×10⁶ to about 1×10¹⁸ vector genomes per kilogram (vg/kg).

26. The method of claim 24, wherein the virus is administered at a dose from about 1×10⁷-1×10¹⁷, about 1×10⁸-1×10¹⁶, about 1×10⁹-1×10¹⁵, about 1×10¹⁰-1×10¹⁴, about 1×10¹⁰-1×10¹³, about 1×10¹⁰-1×10¹³, about 1×10¹⁰-1×10¹¹, about 1×10¹¹-1×10¹², about 1×10¹²-1×10¹³, or about 1×10¹³-1×10¹⁴ vg/kg of the patient.

27. The method of claim 1, wherein the subject is human.

28. The method of claim 1, wherein the subject is a non-human mammal.

29. The method of any one of claim 27, wherein the human subject is 50 or more years old.

30. The method of claim 1, wherein the therapeutic transgene is linked to a poly-adenylation signal.

31. A modified adeno-associated virus (AAV) encoding a therapeutic transgene operably linked to a promoter selected from the group consisting of SEQ ID NO: 1, SEQ ID NO: 2, SEQ ID NO: 3, or a promoter having at least about 80% sequence identity therewith.

32. The modified AAV of claim 31, wherein the promoter is SEQ ID NO: 1 or a promoter having at least about 85%, 90%, 95%, 96%, 97%, 98% or 99% sequence identity therewith.

33. The modified AAV of claim 31, wherein the promoter is SEQ ID NO: 2 or a promoter having at least about 85%, 90%, 95%, 96%, 97%, 98% or 99% sequence identity therewith.

34. The modified AAV of claim 31, wherein the promoter is SEQ ID NO: 3 or a promoter having at least about 85%, 90%, 95%, 96%, 97%, 98% or 99% sequence identity therewith.

35. The modified AAV of claim 31, wherein the modified AAV comprises a modified capsid protein.

36. The modified AAV of claim 35, wherein the modified capsid protein comprises a targeting peptide, wherein the targeting peptide is three to ten amino acids in length, such as seven amino acids in length.

37. The modified AAV of claim 35, wherein the modified AAV capsid protein is a modified AAV1 capsid protein, a modified AAV2 capsid protein, or a modified AAV9 capsid protein.

38. The modified AAV of claim 37, wherein the modified AAV capsid protein is derived from an AAV1 capsid protein, wherein the targeting peptide is inserted after residue 590 of the AAV1 capsid protein.

39. The modified AAV of claim 38, wherein the targeting peptide is flanked by linker sequences, wherein the linker sequences on each side of the targeting peptides are two or three amino acids long.

40. The modified AAV of claim 39, wherein the linker sequences are SSA on the N-terminal side of the targeting peptide and AS on the C-terminal side of the targeting peptide.

41. The modified AAV of claim 37, wherein the modified AAV capsid protein is derived from an AAV2 capsid protein, wherein the targeting peptide is inserted after residue 587 of the AAV2 capsid protein.

42. The modified AAV of claim 41, wherein the targeting peptide is flanked by linker sequences, wherein the linker sequences on each side of the targeting peptides are two or three amino acids long.

43. The modified AAV of claim 42, wherein the linker sequences are AAA on the N-terminal side of the targeting peptide and AA on the C-terminal side of the targeting peptide.

44. The modified AAV of claim 37, wherein the modified AAV capsid protein is derived from an AAV9 capsid protein, wherein the targeting peptide is inserted after residue 588 of the AAV9 capsid protein.

45. The modified AAV of claim 44, wherein the targeting peptide is flanked by linker sequences, wherein the linker sequences on each side of the targeting peptides are two or three amino acids long.

46. The modified AAV of claim 45, wherein the linker sequences are AAA on the N-terminal side of the targeting peptide and AS on the C-terminal side of the targeting peptide.

47. The modified AAV of claim 31, wherein the therapeutic transgene is an siRNA, shRNA, miRNA, non-coding RNA, lncRNA, therapeutic protein, or CRISPR system.

48. The modified AAV of claim 31, wherein the therapeutic transgene is linked to a poly-adenylation signal.

49. The modified AAV of claim 31, wherein the therapeutic transgene is transcriptionally linked to a detectable reporter, e.g., sequence encoding a fluorescent protein, a peptide tag, or a luciferase.

50. A pharmaceutical composition comprising the modified AAV of claim 31 and a pharmaceutically acceptable carrier.

51. An isolated and purified nucleic acid comprising a sequence selected from the group consisting of SEQ ID NO: 1, SEQ ID NO: 2, SEQ ID NO: 3, or a sequence having at least about 80% sequence identity therewith.

52. The nucleic acid of claim 51, wherein the sequence is SEQ ID NO: 1 or a sequence having at least about 85%, 90%, 95%, 96%, 97%, 98% or 99% sequence identity therewith.

53. The nucleic acid of claim 51, wherein the sequence is SEQ ID NO: 2 or a sequence having at least about 85%, 90%, 95%, 96%, 97%, 98% or 99% sequence identity therewith.

54. The nucleic acid of claim 51, wherein the sequence is SEQ ID NO: 3 or a sequence having at least about 85%, 90%, 95%, 96%, 97%, 98% or 99% sequence identity therewith.

55. The nucleic acid of claim 51, wherein the sequence is operably connected to a heterologous coding region.

56. The nucleic acid of claim 51, further comprising one of more of (a) a multipurpose cloning site, (b) a transcription termination signal, (c) a poly-adenylation sequence, and/or (d) an origin of replication.

57. The nucleic acid of claim 51, further comprising one of more of (a) a sequence encoding a detectable marker, (b) a sequence encoding an affinity tag, and/or (c) one or two adeno-associated virus inverted terminal repeats.

58. The nucleic acid of claim 51, wherein said nucleic acid is contained in a replicable vector.

59. The nucleic acid of claim 51, wherein the therapeutic transgene is transcriptionally linked to a reporter by a sequence encoding a 2A “self-cleaving” peptide.

60. A method of reducing or impairing microglial inflammation comprising delivering ApoE2 to microglia in a subject in need thereof.

61. The method of claim 60, wherein the delivering ApoE2 to the microglial comprises administering to said subject ApoE2 protein or a modified AAV encoding SEQ ID NO: 1, SEQ ID NO: 2, SEQ ID NO: 3, or a promoter having at least about 80% sequence identity therewith, and a therapeutic ApoE2 transgene.

62. The method of claim 60, wherein said microglial inflammation is caused by or associated with a neurodegenerative disease, such as Huntington's disease, Parkinson's disease, motor neuron disease, spinocerebellar ataxia, spinal muscular atrophy, progressive supranuclear palsy, amyotrophic lateral sclerosis, multiple sclerosis, Batten disease, and Creutzfeldt-Jakob disease.

63. The method of claim 60, wherein the administration of ApoE2 or modified AAV is direct intracerebroventricular or intraparenchymal injection.

64. The method of claim 60, wherein the ApoE2 protein or modified AAV is administered more than once.

65. The method of claim 64, wherein the ApoE2 protein or modified AAV is administered 2, 3, 4, 5, 6, 7, 8, 9, 10 or more times.

66. The method of claim 64, wherein the ApoE2 protein or modified AAV is administered monthly, every other month, every three months, every four months, every six months or annually.

67. The method of claim 60, further comprising providing a non-AAV ApoE2 therapy to said subject.

68. The method of claim 60, wherein a plurality of viral particles are administered.

69. The method of claim 68, wherein the AAV is administered at a dose of about 1×10⁶ to about 1×10¹⁸ vector genomes per kilogram (vg/kg).

70. The method of claim 68, wherein the virus is administered at a dose from about 1×10⁷-1×10¹⁷, about 1×10⁸-1×10¹⁶, about 1×10⁹-1×10¹⁵, about 1×10¹⁰-1×10¹⁴, about 1×10¹⁰-1×10¹³, about 1×10¹⁰-1×10¹³, about 1×10¹⁰-1×10¹¹, about 1×10¹¹-1×10¹², about 1×10¹²-1×10¹³, or about 1×10¹³-1×10¹⁴ vg/kg of the patient.

71. The method of claim 60, wherein the subject is human.

72. The method of claim 60, wherein the subject is a non-human mammal.

73. The method of claim 72, wherein the human subject is 50 or more years old.

74. The method of claim 60, wherein the therapeutic transgene is linked to a poly-adenylation signal.

75. The method of claim 60, wherein the microglial inflammation is caused by or associated with Alzheimer's disease.
